(19) United States
(12) Patent Application Publication
(10) Pub. No.: US 2023/0293734 A1
(43) Pub. Date: Sep. 21, 2023

(54) FLUORINE-CONTAINING COMPOUND AND CONTRAST AGENT

(71) Applicant: TDK Corporation, Tokyo (JP)

(72) Inventor: Naoko YANAI, Tokyo (JP)

(73) Assignee: TDK Corporation, Tokyo (JP)

(21) Appl. No.: 18/119,456

(22) Filed: Mar. 9, 2023

(30) Foreign Application Priority Data
Mar. 15, 2022 (JP) 2022-040285

(51) Int. Cl. A61K 51/04; C07B 59/00

(52) U.S. Cl. CPC A61K 51/0455; C07B 59/002; C07B 2200/05

(57) ABSTRACT

The fluorine-containing compound includes: two nitroxide radical-containing groups represented by Formula (1) (R¹, R², R³, and R⁴ are each independently a C1-10 alkyl group unsubstituted or substituted with a substituent containing no fluorine atoms); and one to three —O—C(CF₃)₃ groups, in which the nitroxide radical-containing groups are bound to the —O—C(CF₃)₃ groups through a chain structure with 2 to 17 atoms.

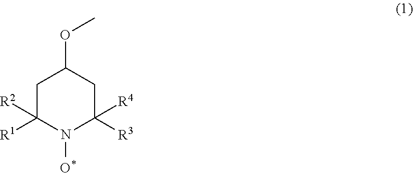

### TECHNICAL FIELD

The present invention relates to a fluorine-containing compound and a contrast agent. Priority is claimed on Japanese Patent Application No. 2022-040285, filed Mar. 15, 2022, the content of which is incorporated herein by reference.

### BACKGROUND ART

Magnetic resonance imaging (hereinafter sometimes referred to as “MRI”) diagnosis is widely used in the medical field for both basic research and clinical application as one of diagnostic imaging methods along with X-ray diagnosis and ultrasound (US) diagnosis.

Currently, ¹H-MRI using protons (¹H) as detection nuclei is used for medical MRI. ¹H-MRI captures and images the magnetic environment of water molecules present in vivo. A difference occurs in the magnetic environment of protons between lesion tissue and normal tissue in vivo. This appears as a difference in ¹H-MRI and serves as diagnostic information. In addition, water molecules are present almost everywhere in vivo. For this reason, ¹H-MRI can be used for whole-body imaging.

Nuclides detectable by MRI include ¹⁹F, ²³Na, ³¹P, ¹⁵N, ¹³C, and the like in addition to ¹H. MRI using these elements as detection nuclei provides information different from ¹H-MRI.

Among these, MRI using ¹⁹F as a detection nucleus is expected to be used as a next-generation diagnostic method following ¹H-MRI diagnosis. This is because fluorine is an inexpensive element with a natural abundance ratio of 100%, detection sensitivity of ¹⁹F is as high as 83% of ¹H, and the gyromagnetic ratio of ¹⁹F is close to that of a proton, so imaging can be performed with a conventional ¹H-MRI device.

In addition, ¹⁹F detectable by MRI is hardly present in vivo. For this reason, by using a fluorine atom-containing compound in a contrast agent, ¹⁹F-MRI diagnosis using ¹⁹F as a tracer is possible. For example, positional information of lesion portions can be obtained from ¹⁹F-MRI using a fluorine compound, which recognizes and accumulates endogenous changes caused by a disease, in a contrast agent. This method is useful for diagnosing lesion portions that do not cause morphological changes that could not be detected by conventional diagnostic imaging methods.

In ¹⁹F-MRI diagnosis, by extracting information such as chemical shift, diffusion, and relaxation time, more diagnostic information can be obtained in addition to the positional information of the lesion portions. In addition, by taking ¹⁹F-MRI and ¹H-MRI simultaneously in one diagnosis and superimposing the images, it is possible to obtain useful diagnostic information in which anatomical information and functional information coexist.

As a fluorine-containing compound used in a contrast agent for MRI diagnosis using fluorine as a detection nucleus, for example, Patent Document 1 discloses a compound having a nitroxide covalently bound to a fluorine-containing compound.

### PATENT DOCUMENTS

[Patent Document 1] U.S. Pat. No. 5,362,477

### SUMMARY OF THE INVENTION

However, high-sensitivity MRI could not be obtained from conventional contrast agents used for MRI diagnosis using fluorine as a detection nucleus.

The present invention has been made in consideration of the above-described circumstances, and an object of the invention is to provide a fluorine-containing compound that can be used as a material for a contrast agent for magnetic resonance imaging diagnosis using fluorine as a detection nucleus to obtain a high-sensitivity magnetic resonance image.

In addition, another object of the present invention is to provide a contrast agent for magnetic resonance imaging diagnosis containing the fluorine-containing compound of the present invention, capable of obtaining a high-sensitivity image, and using fluorine as a detection nucleus.

[1] A fluorine-containing compound including: two nitroxide radical-containing groups represented by General Formula (1) below; and one to three fluorine atom-containing groups represented by Formula (2) below, in which the nitroxide radical-containing groups are bound to the fluorine atom-containing groups through a chain structure with 2 to 17 atoms linked by bonds between carbon atoms or through a chain structure with 2 to 17 atoms linked by an ether bond and bonds between carbon atoms.

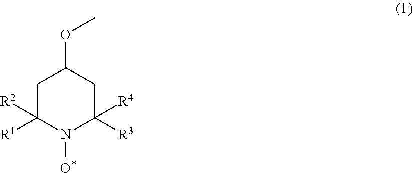

(In General Formula (1), R¹, R², R³, and R⁴ each independently represent a C1-10 alkyl group unsubstituted or substituted with a substituent containing no fluorine atoms)

—O—C(CF₃)₃ (2)

[2] The fluorine-containing compound according to [1], in which two groups each having the nitroxide radical-containing group are bound to an identical carbon atom, and the group having the nitroxide radical-containing group is represented by General Formula (3) below,

—CH₂—(O—(CH₂)ₛ)ₜ—R⁵ (3)

(in General Formula (3), s is an integer of 1 to 12, t is 0 or 1, and R⁵ is represented by General Formula (1) above).

[3] The fluorine-containing compound according to [1] or [2], in which two or three groups each having the fluorine atom-containing group are bound to an identical carbon atom, and the group having the fluorine atom-containing group is represented by Formula (4) below,

—CH₂—O—C(CF₃)₃ (4).

[4] The fluorine-containing compound according to any one of [1] to [3], in which the two groups each having the fluorine atom-containing group are bound to the carbon atom to which the two groups each having the nitroxide radical-containing group are bound.

[5] The fluorine-containing compound according to any one of [1] to [3], in which the carbon atom, to which the two groups each having the nitroxide radical-containing group are bound, is bound to the carbon atom, to which the three groups each having the fluorine atom-containing group are bound, through a linking group represented by Formula (5) below,

—CH₂—O— (5).

[6] The fluorine-containing compound according to any one of [1] to [5], in which R¹, R², R³, and R⁴ in General Formula (1) above each independently represent a methyl group or an ethyl group.

[7] The fluorine-containing compound according to any one of [1] to [6], which is used in a contrast agent for magnetic resonance imaging diagnosis using fluorine as a detection nucleus.

[8] A contrast agent for magnetic resonance imaging diagnosis using fluorine as a detection nucleus, including: the fluorine-containing compound according to any one of [1] to [7].

A fluorine-containing compound of the present invention includes: two nitroxide radical-containing groups represented by General Formula (1) above; and one to three fluorine atom-containing groups represented by Formula (2) above, in which the nitroxide radical-containing groups are bound to the fluorine atom-containing groups through a chain structure with 2 to 17 atoms linked by bonds between carbon atoms or through a chain structure with 2 to 17 atoms linked by an ether bond and bonds between carbon atoms. For this reason, the fluorine-containing compound of the present invention is used as a material for a contrast agent for magnetic resonance imaging diagnosis using fluorine as a detection nucleus to obtain a high-sensitivity magnetic resonance image.

A contrast agent of the present invention contains the fluorine-containing compound of the present invention. For this reason, the contrast agent of the present invention is used as a contrast agent for magnetic resonance imaging diagnosis using fluorine as a detection nucleus to obtain a high-sensitivity magnetic resonance image.

### BRIEF DESCRIPTION OF THE DRAWINGS

FIG. 1 is a ¹⁹F spin-lattice relaxation time (T1)-weighted ¹⁹F-MRI image for Example 1 (compound 11), Example 5 (compound 15), and Comparative Example 1 (compound A1).

FIG. 2 is a ¹⁹F spin-lattice relaxation time (T1)-weighted ¹⁹F-MRI image for Example 1 (compound 11), Example 5 (compound 15), and Comparative Example 1 (compound A1) and a photograph in which positions of Examples 1 and 5 and Comparative Example 1 on the image shown in FIG. 1 are shown.

### DETAILED DESCRIPTION OF THE INVENTION

Hereinafter, a fluorine-containing compound and a contrast agent of the present invention will be described in detail.

### [Fluorine-Containing Compound]

A fluorine-containing compound of the present embodiment includes: two nitroxide radical-containing groups represented by General Formula (1) below; and one to three fluorine atom-containing groups represented by Formula (2) below.

In the fluorine-containing compound of the present embodiment, the nitroxide radical-containing groups are bound to the fluorine atom-containing groups through a chain structure with 2 to 17 atoms. The chain structure with 2 to 17 atoms is a chain structure with 2 to 17 atoms linked by bonds between carbon atoms or a chain structure with 2 to 17 atoms linked by an ether bond (—O—) and bonds between carbon atoms.

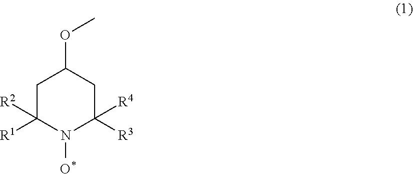

(in General Formula (1), R¹, R², R³, and R⁴ each independently represent a C1-10 alkyl group unsubstituted or substituted with a substituent containing no fluorine atoms)

—O—C(CF₃)₃ (2)

Here, the reason why a high-sensitivity magnetic resonance image (MRI) can be obtained when the contrast agent containing the fluorine-containing compound of the present embodiment is used as a contrast agent for MRI diagnosis using fluorine as a detection nucleus will be described.

In order to obtain high-sensitivity ¹⁹F-MRI, it is preferable to use a fluorine-containing compound with a short ¹⁹F spin-lattice relaxation time (T1) as a fluorine-containing compound contained in a contrast agent. The shorter the T1 of the fluorine-containing compound, the shorter the repetition time can be set. For this reason, the amount of signal obtained per unit time is increased, and a high-sensitivity image can be obtained. On the other hand, if the ¹⁹F spin-spin relaxation time (T2) of the fluorine-containing compound is too short, the signal intensity will decrease.

The ¹⁹F spin-lattice relaxation time (T1) and ¹⁹F spin-spin relaxation time (T2) of a fluorine-containing compound are affected by a paramagnetic relaxation enhancement (PRE) effect. The PRE effect is a phenomenon in which T1 and T2 of MRI observation nuclei in the vicinity of unpaired electron spins possessed by a paramagnetic material are shortened by the unpaired electron spins.

The PRE effect is inversely proportional to the sixth power of the distance between the paramagnetic material and a MRI observation nucleus (a fluorine atom in the present embodiment) relaxed by the paramagnetic material. Accordingly, in the fluorine-containing compound of the present embodiment, the shorter the distance between a fluorine atom and a nitroxide radical which is a paramagnetic material, the shorter T1 and T2 are.

In the fluorine-containing compound of the present embodiment, the nitroxide radical-containing groups represented by Formula (1) are bound to the fluorine atom-containing groups represented by Formula (2) through a chain structure with 2 to 17 atoms. For this reason, the distance between a nitroxide radical and a fluorine atom is appropriate, T1 is sufficiently short, and T2 can be sufficiently secured. Accordingly, the fluorine-containing compound of the present embodiment is used in a contrast agent for MRI diagnosis using fluorine as a detection nucleus to obtain a high-sensitivity magnetic resonance image.

In addition, in order to obtain high-sensitivity ¹⁹F-MRI, it is preferable to use a fluorine-containing compound with a large number of structurally equivalent fluorine atoms as a fluorine-containing compound contained in a contrast agent. The fluorine-containing compound of the present embodiment has one to three fluorine atom-containing groups represented by Formula (2). The fluorine atom-containing group represented by Formula (2) contains three trifluoromethyl groups (—CF₃). The trifluoromethyl group (—CF₃) contains three structurally equivalent fluorine atoms. Accordingly, the fluorine-containing compound of the present embodiment has 9 to 27 structurally equivalent fluorine atoms in a molecule. Accordingly, in a case where the fluorine-containing compound of the present embodiment which has a sufficiently large number of structurally equivalent fluorine atoms contained in a molecule is used in a contrast agent for MRI diagnosis using fluorine as a detection nucleus, a strong signal intensity and a high-sensitivity image are obtained.

Moreover, the fluorine-containing compound of the present embodiment has two nitroxide radical-containing groups represented by General Formula (1). For this reason, in the case where the fluorine-containing compound of the present embodiment is used in a contrast agent for MRI diagnosis using fluorine as a detection nucleus, the ¹⁹F spin-lattice relaxation time (T1) becomes shorter than in a case where the number of nitroxide radical-containing groups is, for example, only one, so that the repetition time can be set to be short. For this reason, the amount of signal obtained per unit time can be increased, and a high-sensitivity image can be obtained.

R¹, R², R³, and R⁴ in the nitroxide radical-containing group represented by Formula (1) are each independently a C1-10 alkyl group unsubstituted or substituted with a substituent containing no fluorine atoms and preferably a C1-5 alkyl group unsubstituted or substituted with a substituent containing no fluorine atoms. Since R¹, R², R³, and R⁴ are each independently a substituted or unsubstituted C1-10 alkyl group, the fluorine-containing compound of the present embodiment is easily synthesized. In addition, if R¹, R², R³, and R⁴ are substituted or unsubstituted C2-10 alkyl groups, they become appropriately bulky and can prevent the approach of a reducing agent to the nitroxide radical. If the number of carbon atoms in the above-described alkyl group is 5 or less, synthesis of the fluorine-containing compound of the present embodiment becomes much easier, which is preferable. In addition, since R¹, R², R³, and R⁴ do not contain a fluorine atom in the fluorine-containing compound of the present embodiment, there is no case where the signal intensity is insufficient due to a too close distance between the nitroxide radical and a fluorine atom.

In a case where R¹, R², R³, and R⁴ contained in the nitroxide radical-containing group represented by Formula (1) have a substituent containing no fluorine atoms, a methyl group or an ethyl group can be used as the substituent, for example.

Specifically, it is preferable that R¹, R², R³, and R⁴ in the nitroxide radical-containing group represented by Formula (1) be each independently a methyl group or an ethyl group. In a case where R₁, R², R³, and R⁴ are methyl groups, the number of production steps can be reduced compared to a case where they are ethyl groups, and a fluorine-containing compound having excellent productivity is obtained, which is preferable.

R¹, R², R³, and R⁴ contained in the nitroxide radical-containing group represented by Formula (1) may be different from each other, or some or all of them may be the same as each other. In a case where R¹, R², R³, and R⁴ are all the same, the fluorine-containing compound represented by Formula (1) is easily synthesized, which is preferable.

The fluorine-containing compound of the present embodiment has one to three fluorine atom-containing groups represented by Formula (2). Accordingly, in a case where the fluorine-containing compound of the present embodiment which has a sufficiently large number of structurally equivalent fluorine atoms indicating a ¹⁹F-MRI peak is used in a contrast agent for MRI diagnosis using fluorine as a detection nucleus, a strong signal intensity and a high-sensitivity image are obtained.

The number of fluorine atom-containing groups represented by Formula (2) in the fluorine-containing compound of the present embodiment is 1 to 3, preferably 2 or 3, and more preferably 3. This is because the number of structurally equivalent fluorine atoms increases as the number of trifluoromethyl groups (—CF₃) contained in the fluorine-containing compound increases. In three-dimensional (3D) MRI, the greater the number of fluorine atoms in a voxel (in a pixel in two-dimensional (2D) MRI), the greater the amount of signal obtained and the more high-sensitivity images are likely to be obtained, which is preferable.

In the fluorine-containing compound of the present embodiment, the nitroxide radical-containing groups represented by Formula (1) are bound to the fluorine atom-containing groups represented by Formula (2) through a chain structure with 2 to 17 atoms. For this reason, the distance between a nitroxide radical and a fluorine atom is appropriate, the ¹⁹F spin-lattice relaxation time (T1) is sufficiently short, and the ¹⁹F spin-spin relaxation time (T2) can be sufficiently secured. Accordingly, the fluorine-containing compound of the present embodiment is used in a contrast agent for MRI diagnosis using fluorine as a detection nucleus to obtain a high-sensitivity magnetic resonance image. The number of atoms in the above-described chain structure is preferably 2 to 10 and more preferably 2 to 6 so that the distance between a nitroxide radical and a fluorine atom becomes more appropriate.

In the fluorine-containing compound of the present embodiment, the chain structure with 2 to 17 atoms through which the nitroxide radical-containing groups are bound to the fluorine atom-containing groups is a chain structure with 2 to 17 atoms linked by bonds between carbon atoms or a chain structure with 2 to 17 atoms linked by an ether bond (—O—) and bonds between carbon atoms.

The number of atoms in the chain structure with 2 to 17 atoms linked by bonds between carbon atoms is preferably 2 to 6 and more preferably 2 or 3. This is because the fluorine-containing compound can be easily produced and has excellent productivity.

The number of atoms in the chain structure with 2 to 17 atoms linked by an ether bond (—O—) and bonds between carbon atoms is preferably 6 to 17 and more preferably 6 to 12. This is because the distance between a nitroxide radical and a fluorine atom is appropriate.

In the fluorine-containing compound of the present embodiment, two groups each having the nitroxide radical-containing group represented by Formula (1) are preferably bound to an identical carbon atom. This is because the fluorine-containing compound can be easily produced and has excellent productivity.

The carbon atom to which the two groups each having the nitroxide radical-containing group are bound is an atom constituting a part of the chain structure through which the nitroxide radical-containing groups are bound to the fluorine atom-containing groups.

In addition to the two groups each having the nitroxide radical-containing group, a group forming a side chain of the above-described chain structure may be bound to the carbon atom to which the two groups each having the nitroxide radical-containing group are bound. The group forming a side chain of the above-described chain structure may be a group containing no fluorine atom, and is preferably a C1-5 alkyl group and more preferably a methyl group or an ethyl group to facilitate production.

The group having the nitroxide radical-containing group may be a group having the nitroxide radical-containing group represented by Formula (1), and is preferably a group in which one or a plurality of ether bonds (—O—) and/or methylene groups (—CH₂—) are bound to the nitroxide radical-containing group represented by Formula (1) and more preferably the group represented by General Formula (3).

—CH₂—(O—(CH₂)ₛ)ₜ—R⁵ (3)

(In Formula (3), s is an integer of 1 to 12, t is 0 or 1, and R⁵ is represented by Formula (1) above.)

In the group which has the nitroxide radical-containing group and is represented by Formula (3), s is an integer of 1 to 12 and preferably an integer of 1 to 6. This is because a fluorine-containing compound having a more appropriate distance between a nitroxide radical and a fluorine atom is likely to be obtained.

In the group which has the nitroxide radical-containing group and is represented by Formula (3), t is 0 or 1 and can be appropriately determined depending on, for example, the raw material and production method of the fluorine-containing compound and on the distance between target nitroxide radical and fluorine atom.

The two groups each having the nitroxide radical-containing group represented by Formula (1) contained in the fluorine-containing compound may be the same as or different from each other, and preferably the same as each other. This is because the fluorine-containing compound can be easily produced and has excellent productivity. In addition, in a case where the two groups each having the nitroxide radical-containing group are the same as each other and bound to an identical carbon atom and there is only one fluorine atom-containing group represented by Formula (2), in a molecule, the distances between nitroxide radicals and fluorine atoms become uniform, and a fluorine-containing compound having more accurate distances between nitroxide radicals and fluorine atoms is likely to be obtained, which is preferable.

In the fluorine-containing compound of the present embodiment, two or three groups each having the fluorine atom-containing group represented by Formula (2) are preferably bound to an identical carbon atom. This is because even in a case where the fluorine-containing compound has two or three groups each having the fluorine atom-containing group, the fluorine-containing compound can be easily produced in a small number of production steps and has favorable productivity.

The carbon atom to which the two or three groups each having the fluorine atom-containing group are bound is an atom constituting a part of the chain structure through which the nitroxide radical-containing groups are bound to the fluorine atom-containing groups.

In the case where the fluorine-containing compound has two or three groups each having the fluorine atom-containing group, groups each having the fluorine atom-containing group may be different from each other, or some or all of them may be the same as each other, and all of them are preferably the same as each other. This is because the fluorine-containing compound can be easily produced in a small number of production steps and has excellent productivity. This is because, if the two or three groups each having the fluorine atom-containing group are all the same as each other, in a case where the fluorine-containing compound which has a sufficiently large number of structurally equivalent fluorine atoms indicating a ¹⁹F-MRI peak is used in a contrast agent for MRI diagnosis using fluorine as a detection nucleus, a strong signal intensity and a high-sensitivity image are obtained.

In the case where the fluorine-containing compound has two or three groups each having the fluorine atom-containing group, the groups each having the fluorine atom-containing group may have the fluorine atom-containing group represented by Formula (2) and can be appropriately determined depending on, for example, the raw material and production method of the fluorine-containing compound and on the distance between target nitroxide radical and fluorine atom. Specifically, the groups each having the fluorine atom-containing group may be a group in which one or a plurality of ether bonds (—O—) and/or methylene groups (—CH₂—) are bound to the fluorine atom-containing group represented by Formula (2) and more preferably the group represented by Formula (4).

—CH₂—O—C(CF₃)₃ (4)

In the fluorine-containing compound of the present embodiment, it is preferable that the two groups each having the nitroxide radical-containing group represented by Formula (1) be the same as each other and bound to an identical carbon atom and that the two or three groups each having the fluorine atom-containing group represented by Formula (2) be the same as each other and bound to an identical carbon atom. This is because, in this case, in a molecule, the distances between nitroxide radicals contained in the two groups each having the nitroxide radical-containing group and fluorine atoms contained in the two or three groups each having the fluorine atom-containing group become uniform, and a fluorine-containing compound having more accurate distances between nitroxide radicals and fluorine atoms is likely to be obtained, which is preferable.

In the present embodiment, in a case where the two groups each having the nitroxide radical-containing group are bound to an identical carbon atom and the two groups each having the fluorine atom-containing group are bound to an identical carbon atom, the two groups each having the fluorine atom-containing group are preferably bound to the carbon atom to which the two groups each having the nitroxide radical-containing group are bound. This is because the fluorine-containing compound can be easily produced in a small number of production steps and has excellent productivity.

In the present embodiment, in a case where the two groups each having the nitroxide radical-containing group are bound to an identical carbon atom and the three groups each having the fluorine atom-containing group are bound to an identical carbon atom, the carbon atom, to which the two groups each having the nitroxide radical-containing group are bound, is preferably bound to the carbon atom, to which the three groups each having the fluorine atom-containing group are bound, through a linking group represented by Formula (5) below. This is because the fluorine-containing compound can be easily produced and has excellent productivity.

—CH₂—O— (5)

—CH₂— in Formula (5) may be bound to the carbon atom to which the three groups each having the fluorine atom-containing group are bound or may be bound to the carbon atom to which the two groups each having the nitroxide radical-containing group are bound, and is preferably bound to the carbon atoms to which the three groups each having the fluorine atom-containing group are bound to obtain a fluorine-containing compound that can be easily produced and has excellent productivity.

Specifically, the fluorine-containing compound of the present embodiment is preferably any one of fluorine-containing compounds represented by Formulae (11) to (23) below. In a case where all of the compounds represented by Formulae (11) to (23) are used in contrast agents for MRI diagnosis using fluorine as a detection nucleus, high-sensitivity images are obtained.

In the compounds represented by Formulae (11) to (14), two groups, each of which has the fluorine atom-containing group and is represented by Formula (4), are bound to a carbon atom to which two groups, each of which has the nitroxide radical-containing group and is represented by Formula (3), are bound. The number of atoms in a chain structure through which the nitroxide radical-containing groups represented by Formula (1) are bound to the fluorine atom-containing groups represented by Formula (2) is 3 in the compound represented by Formula (11), 3 in the compound represented by Formula (12), 7 in the compound represented by Formula (13), and 10 in the compound represented by Formula (14).

The compounds represented by Formulae (15) to (19) have three groups, each of which has the fluorine atom-containing group and is represented by Formula (4). In the compounds represented by Formulae (15) to (19), a carbon atom, to which two groups, each of which has the nitroxide radical-containing group and is represented by Formula (3), are bound, is bound to a carbon atom, to which three groups having the fluorine atom-containing group are bound, through a linking group represented by

Formula (5). The number of atoms in a chain structure through which the nitroxide radical-containing groups represented by Formula (1) are bound to the fluorine atom-containing groups represented by Formula (2) is 6 in the compound represented by Formula (15), 6 in the compound represented by Formula (16), 6 in the compound represented by Formula (17), 10 in the compound represented by Formula (18), and 13 in the compound represented by Formula (19).

The compounds represented by Formulae (20) to (23) have one group having the fluorine atom-containing group represented by Formula (2). The number of atoms in a chain structure through which the nitroxide radical-containing groups represented by Formula (1) are bound to the fluorine atom-containing groups represented by Formula (2) is 2 in the compound represented by Formula (20), 2 in the compound represented by Formula (21), 6 in the compound represented by Formula (22), and 9 in the compound represented by Formula (23).

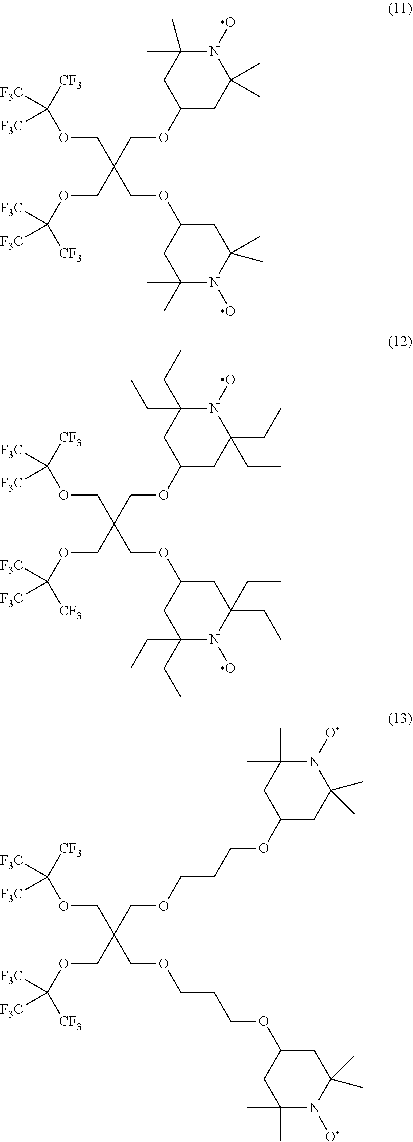

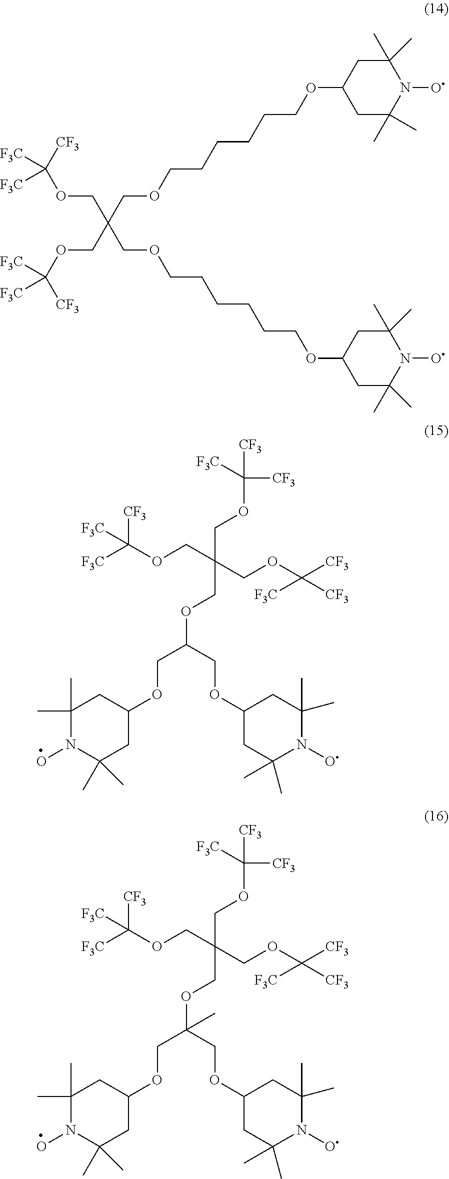

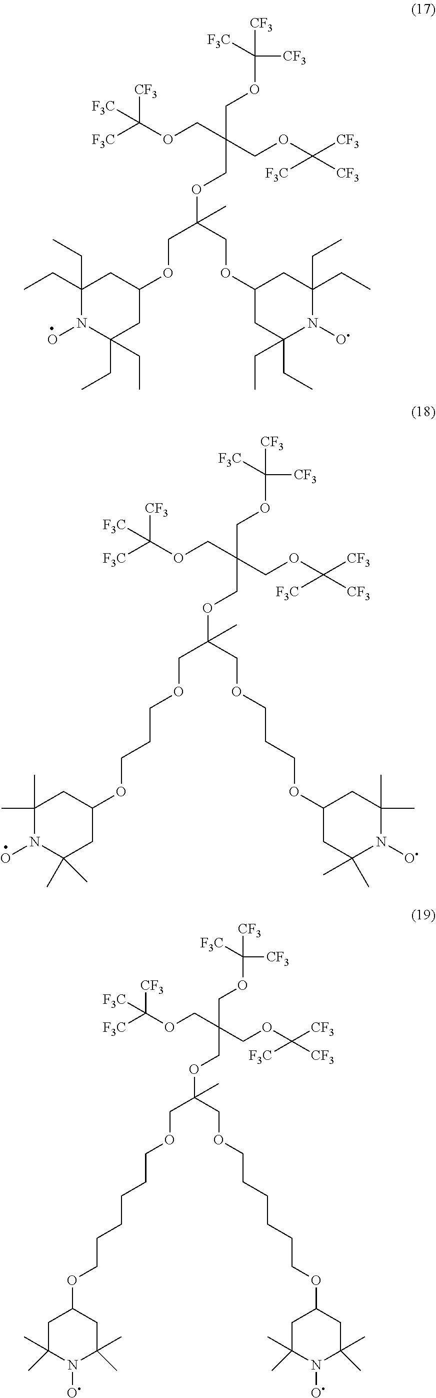

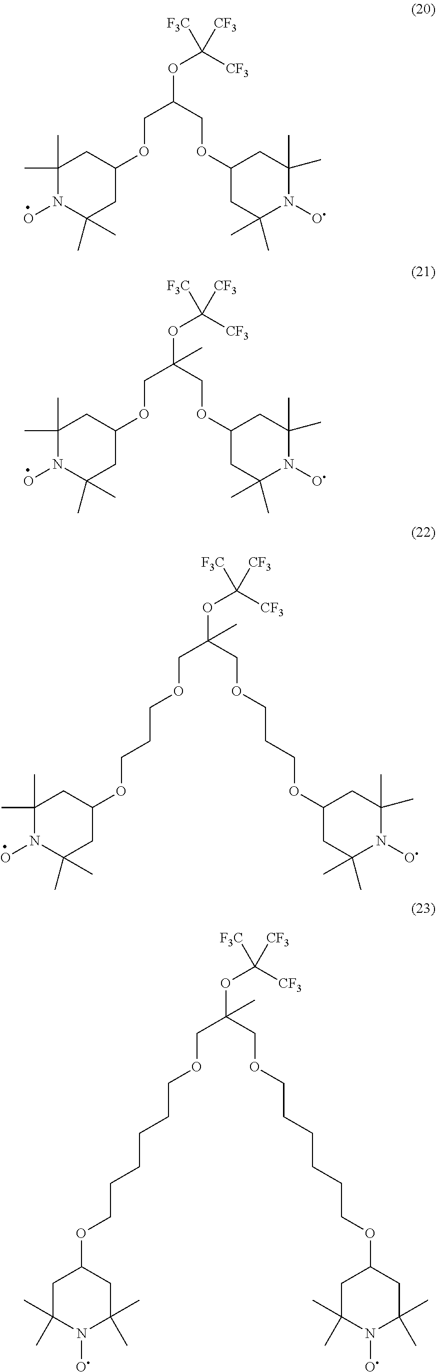

### [Method for Producing Fluorine-Containing Compound]

Next, a method for producing a fluorine-containing compound of the present embodiment will be described as an example.

The method for producing a fluorine-containing compound of the present embodiment is not particularly limited, and the fluorine-containing compound can be produced using a well-known conventional production method.

The fluorine-containing compound of the present embodiment can be produced using, for example, a production method shown below.

### (First Production Method)

<In case where fluorine-containing compound has two groups, each of which has nitroxide radical-containing group and is represented by Formula (3), and two groups, each of which has fluorine atom-containing group and is represented by Formula (4), and two groups each having fluorine atom-containing group are bound to carbon atom to which two groups each having nitroxide radical-containing group are bound>

First, a compound is prepared which has a nitroxide radical and in which R¹, R², R³, and R⁴ in a nitroxide radical-containing group represented by Formula (1) are bound to the 2-position and the 6-position of a piperidine ring and H—(O—(CH₂)ₛ)ₜ—O— (s and tin the formula are the same as those in Formula (3)) is bound to the 4-position. This compound may be synthesized, or a commercially available one may be prepared.

In the case of synthesizing the compound, a method for synthesizing the compound using 2,2,6,6-tetramethyl-4-piperidone as a raw material through a well-known method can be used.

In a case of producing a fluorine-containing compound in which tin the group which has the nitroxide radical-containing group and is represented by Formula (3) is 1, a compound in which the number of methylene groups (—(CH₂)—) bound to the 4-position of the piperidine ring corresponds to the number of s's in Formula (3) in the fluorine-containing compound to be synthesized is used as the above-described compound having a nitroxide radical.

The above-described compound having a nitroxide radical used in the case of producing a fluorine-containing compound in which tin the group which has the nitroxide radical-containing group and is represented by Formula (3) is 1 can be synthesized, for example, using a method shown below.

That is, a tetrahydropyranyl ether having a group represented by —O—(CH₂)ₛ-u (u in the formula is a halogeno group, and s is the same as that in Formula (3)) at the 2-position of tetrahydropyran is used. This compound can be produced through a well-known method. Next, a first intermediate compound is obtained by reacting the halogeno group of the above-described tetrahydropyranyl ether with a hydroxyl group of a compound having a nitroxide radical in which R¹, R², R³, and R⁴ in a nitroxide radical-containing group represented by Formula (1) are bound to the 2-position and the 6-position of a piperidine ring and the hydroxyl group is bound to the 4-position of a piperidine ring. Thereafter, tetrahydropyran is removed from the first intermediate compound through a well-known method. As a result, a compound which has a nitroxide radical and in which a HO—(CH₂)ₛ—O— (s in the formula is the same as that in Formula (3)) group is bound to the 4-position of the piperidine ring. Protection and deprotection of a hydroxyl group using a tetrahydropyranyl group can be carried out using a well-known method.

In addition, a halogen compound having two groups, each of which has a fluorine atom-containing group and is represented by Formula (4), and two halogenated alkyl groups (u-(CH₂)— (u in the formula is a halogeno group)) is prepared. This halogen compound can be produced through a well-known method. For example, it can be produced through a method of reacting nonafluoro-tert-butanol with a compound having two hydroxymethyl groups (—CH₂OH) and two halogenated alkyl groups.

Thereafter, the two halogeno groups in the above-described halogen compound are reacted with the hydroxyl group of the above-described compound having a nitroxide radical.

By the above-described method, a fluorine-containing compound is obtained which has two groups, each of which has a nitroxide radical-containing group and is represented by Formula (3), and two groups, each of which has a fluorine atom-containing group and is represented by Formula (4), and in which the two groups each having the fluorine atom-containing group are bound to a carbon atom to which the two groups each having the nitroxide radical-containing group are bound.

### (Second Production Method)

<In case where fluorine-containing compound has two groups, each of which has nitroxide radical-containing group and is represented by Formula (3), and three groups, each of which has fluorine atom-containing group and is represented by Formula (4), and carbon atom, to which two groups each having nitroxide radical-containing group are bound, is bound to carbon atom, to which three groups each having fluorine atom-containing group are bound, through linking group represented by Formula (5)>

Similarly to the first production method, a compound is prepared which has a nitroxide radical and in which R¹, R², R³, and R⁴ in a nitroxide radical-containing group represented by Formula (1) are bound to the 2-position and the 6-position of a piperidine ring and H—(O—(CH₂)ₛ)ₜ—O— (s and tin the formula are the same as those in Formula (3)) is bound to the 4-position.

In addition, a hydroxyl group of a monovalent secondary alcohol having two halogenated methyl groups is protected using a 2-tetrahydropyranyl group, and the two halogenated methyl groups are reacted with the hydroxyl group of the above-described compound having a nitroxide radical to obtain a second intermediate compound.

Thereafter, tetrahydropyran is removed from the second intermediate compound, and a third intermediate compound which is a monovalent secondary alcohol having two groups, each of which has a nitroxide radical-containing group and is represented by Formula (3), is obtained.

In a case of producing a fluorine-containing compound in which a group forming a side chain of a chain structure is bonded, in addition to the two groups each having the nitroxide radical-containing group, to a carbon atom to which the two groups each having the nitroxide radical-containing group are bound, a monovalent tertiary alcohol in which two halogenated methyl groups are bound to the group forming a side chain of a chain structure is used instead of the monovalent secondary alcohol having two halogenated methyl groups as a raw material of the second intermediate compound. As a result, a monovalent tertiary alcohol having two groups, each of which has a nitroxide radical-containing group and is represented by Formula (3), is obtained as a third intermediate compound.

In addition, a halogen compound having three groups, each of which has a fluorine atom-containing group and is represented by Formula (4), and one halogenated methyl group is prepared. This halogen compound can be produced through a well-known method. For example, it can be produced through a method of reacting nonafluoro-tert-butanol with a compound having three hydroxymethyl groups (—CH₂OH) and a halogenated methyl group.

Thereafter, the halogenated methyl group in the above-described halogen compound is reacted with a hydroxyl group of the above-described third intermediate compound.

By the above-described method, a fluorine-containing compound is obtained which has two groups, each of which has a nitroxide radical-containing group and is represented by Formula (3), and three groups, each of which has a fluorine atom-containing group and is represented by Formula (4), and in which a carbon atom, to which the two groups each having the nitroxide radical-containing group are bound, is bound to a carbon atom, to which the three groups each having the fluorine atom-containing group are bound, through a linking group represented by Formula (5).

### (Third Production Method)

<In case where fluorine-containing compound has two groups, each of which has nitroxide radical-containing group and is represented by Formula (3), and one group having fluorine atom-containing group represented by Formula (2), and group having fluorine atom-containing group is bound to carbon atom to which two groups each having nitroxide radical-containing group are bound>

Similarly to the first production method, a compound having a nitroxide radical in which R¹, R², R³, and R⁴ in a nitroxide radical-containing group represented by Formula (1) are bound to the 2-position and the 6-position of a piperidine ring and H—(O—(CH₂)ₛ)ₜ—O— (s and tin the formula are the same as those in Formula (3)) is bound to the 4-position of a piperidine ring is prepared.

In addition, a fourth intermediate compound is prepared in which two halogenated methyl groups are bound to a carbon atom to which one group having a fluorine atom-containing group represented by Formula (2) is bound. The fourth intermediate compound can be produced through a well-known method. For example, it can be produced through a method of reacting nonafluoro-tert-butanol with a monovalent secondary alcohol having two halogenated methyl groups.

In a case of producing a fluorine-containing compound in which a group forming a side chain of a chain structure is bonded, in addition to the two groups each having the nitroxide radical-containing group, to a carbon atom to which the two groups each having the nitroxide radical-containing group are bound, a monovalent tertiary alcohol in which two halogenated methyl groups are bound to the group forming a side chain of a chain structure is used instead of the monovalent secondary alcohol having two halogenated methyl groups as a raw material of the fourth intermediate compound. As a result, a compound in which two halogenated methyl groups and a group forming a side chain of a chain structure are bound to a carbon atom to which one group having a fluorine atom-containing group represented by Formula (2) is bound is obtained as the fourth intermediate compound.

Thereafter, the halogenated methyl groups in the above-described fourth intermediate compound are reacted with the hydroxyl group of the above-described compound having a nitroxide radical.

By the above-described method, a fluorine-containing compound is obtained which has two groups, each of which has a nitroxide radical-containing group and is represented by Formula (3), and one group having a fluorine atom-containing group represented by Formula (2) and in which the group having a fluorine atom-containing group is bound to a carbon atom to which the two groups each having the nitroxide radical-containing group are bound.

### “Contrast Agent”

A contrast agent of the present embodiment contains a fluorine-containing compound of the present embodiment. The contrast agent of the present embodiment is a contrast agent for magnetic resonance imaging diagnosis using fluorine as a detection nucleus.

The contrast agent of the present embodiment can be produced, for example, through a method for formulating the fluorine-containing compound of the present embodiment into forms such as a solid formulation, a powder preparation, and a liquid formulation using a well-known formulation technique.

The contrast agent of the present embodiment may contain one kind or two or more kinds of additives, such as an excipient, a stabilizer, a surfactant, a buffer agent, and an electrolyte, used in well-known formulations as necessary in addition to the fluorine-containing compound of the present embodiment.

Since the contrast agent of the present embodiment contains the fluorine-containing compound of the present invention, it is used as a contrast agent for magnetic resonance imaging diagnosis using fluorine as a detection nucleus to obtain a high-sensitivity magnetic resonance image.

The embodiment of the present invention has been described. However, the configuration or the like in the above-described embodiment is merely an example, and addition, omission, replacement, and other modifications of the configuration can be made within the scope not departing from the gist of the present invention.

### EXAMPLES

### Example 1

### (Synthesis of Compound 11)

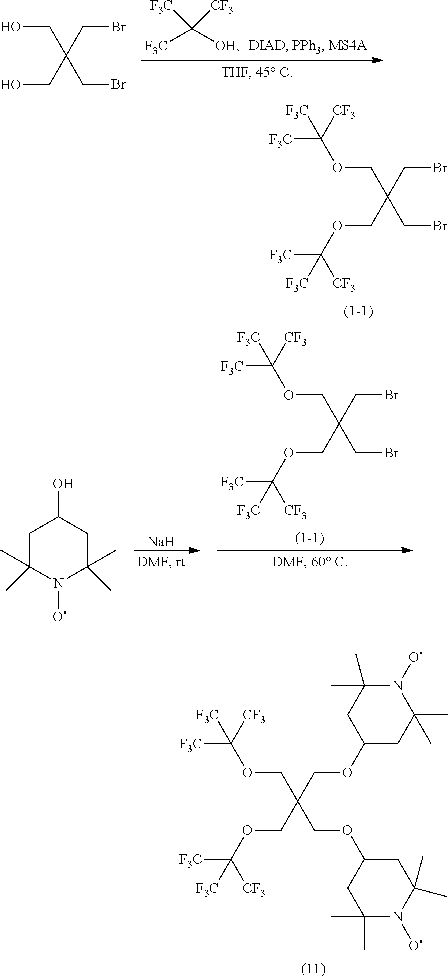

### Synthesis of 2-(3-bromo-2-(bromomethyl)-2-(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-1)

In an argon stream, 11.096 g (42.4 mmol) of 2,2-bis(bromomethyl)-1,3-propanediol, 27.777 g (105.9 mmol) of triphenylphosphine (PPh₃), 10.000 g of molecular sieves (MS) 4 A, and 150 mL of tetrahydrofuran (THF) were mixed with each other and cooled in an ice bath. 20.6 mL (105.9 mmol) of diisopropyl azodicarboxylate (DIAD) was added dropwise thereto over 10 minutes, and the mixture was stirred for 20 minutes. 25.000 g (105.9 mmol) of nonafluoro-tert-butanol was added thereto at one time, and the mixture was stirred at 45° C. for 72 hours.

After the reaction solution was filtered, it was concentrated under reduced pressure and purified through silica gel column chromatography (hexane:ethyl acetate=9:1) to obtain a target compound 2-(3-bromo-2-(bromomethyl)-2-(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-1) (yield of 25.052 g, percent yield of 85%).

<Synthesis of compound (11)>

In an argon stream, 20 mL of dimethylformamide (DMF) was added to 2.723 g (62.4 mmol) of 55% sodium hydride (NaH), and the mixture was stirred at room temperature for 10 minutes. 50 mL of a dimethylformamide solution containing 10.748 g (62.4 mmol) of 4-hydroxy-2,2,6,6-tetramethylpiperidine-1-oxyl was added dropwise thereto over 10 minutes, and the mixture was stirred at room temperature for 3 hours.

80 mL of a dimethylformamide solution containing 16.752 g (24.0 mmol) of 2-(3-bromo-2-(bromomethyl)-2-(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-1) synthesized through the above-described reaction was added thereto in an ice bath, and the mixture was stirred at 60° C. for 14 hours.

Water was added to the reaction solution and extraction was performed with ethyl acetate, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (11) (yield of 9.957 g, percent yield of 47%).

When the obtained compound was subjected to mass spectrometry, a peak was confirmed at m/z=880 (M⁺). From this, it was confirmed that the synthesized compound is a compound represented by Formula (11). In addition, the purity of the compound represented by Formula (11) which was conformed through high-performance liquid chromatography (HPLC) was 99.0%.

### Example 2

### (Synthesis of Compound 12)

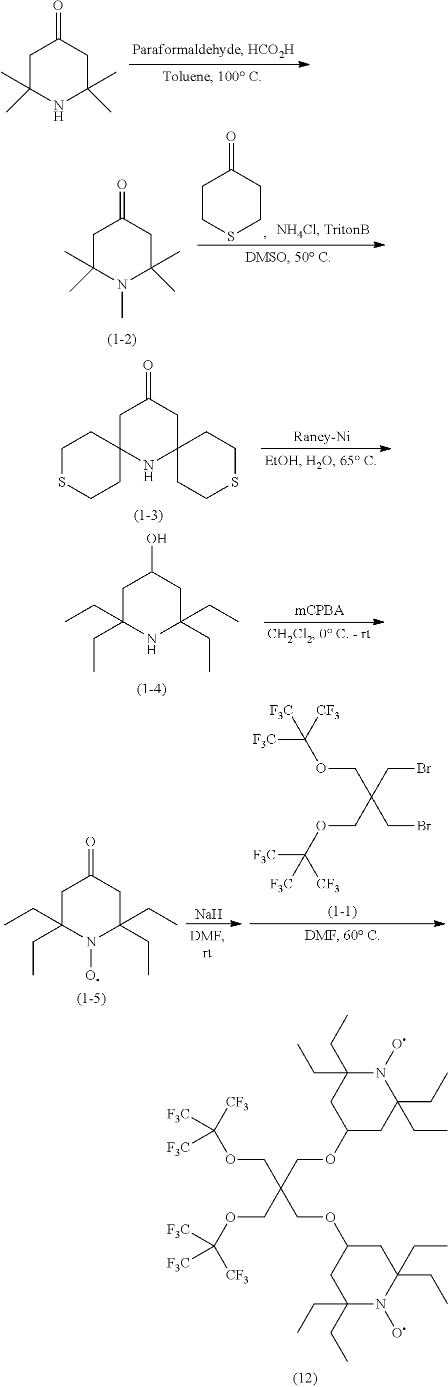

### Synthesis of 1,2,2,6,6-pentamethyl-4-piperidone (1-2)

In an argon stream, 15.524 g (100 mmol) of 2,2,6,6-tetramethyl-4-piperidone, 23.288 g (150 mmol) of paraformaldehyde, and 100 mL of toluene were mixed with each other and heated to 90° C. 5.70 mL (150 mmol) of formic acid was added dropwise thereto over 30 minutes, and the mixture was heated at 100° C. for 12 hours. After cooling to room temperature, 2.000 g (50 mmol) of sodium hydroxide was added thereto, and the mixture was stirred for 1 hour. Thereafter, suction filtration was performed, and the filtrate was concentrated under reduced pressure. The obtained concentrate was distilled under reduced pressure (70-72° C./2 mmHg) to obtain target 1,2,2,6,6-pentamethyl-4-piperidone (1-2) (yield of 13.532 g, percent yield of 80%).

### Synthesis of 7-aza-3.11-dithiadispiro[5.1.5.3]hexadecan-15-one (1-3)

In an argon stream, 12.373 g (73.1 mmol) of 1,2,2,6,6-pentamethyl-4-piperidone (1-2) synthesized through the above-described reaction and 25.000 g (215.2 mmol) of 4-oxothiane were dissolved in 100 mL of dimethyl sulfoxide (DMSO). 23.001 g (430.0 mmol) of ammonium chloride and then 14 mL of 40% benzyltrimethylammonium hydroxide aqueous solution (Triron B) were added thereto, and the mixture was stirred at 50° C. for 10 hours.

After water was added to the reaction solution and the pH was adjusted to 1 with 5% hydrochloric acid, the resultant was washed with diethyl ether. The pH of the water layer was adjusted to 9 with a 10% potassium carbonate aqueous solution, and extraction was performed with ethyl acetate. The organic layer was washed with a saturated sodium chloride aqueous solution and dried with magnesium sulfate, and then concentrated under reduced pressure. The resultant was purified through silica gel column chromatography (hexane:ethyl acetate=3:1 to 1:1) and then reprecipitated with hexane-ethyl acetate to obtain target 7-aza-3.11-dithiadispiro[5.1.5.3]hexadecan-15-one (1-3) (yield of 5.771 g, percent yield of 29%).

### Synthesis of 4-hydroxy-2,2,6,6-tetraethylpiperidine (1-4)

320 mL of ethanol (EtOH) was added to 5.500 g (20.3 mmol) of 7-aza-3.11-dithiadispiro[5.1.5.3]hexadecan-15-one (1-3) synthesized through the above-described reaction in an argon stream. 50.00 g of Raney-Ni (water suspension, Ni>92.5%, A1<6.5%) was added thereto while being washed with 60 mL of ethanol, and the mixture was stirred at 65° C. for 72 hours.

The reaction solution was filtered with Celite and concentrated under reduced pressure. The pH of the resultant was adjusted to 12 with a saturated sodium chloride aqueous solution, extraction was performed with ethyl acetate, and the extract was washed with a saturated sodium chloride aqueous solution. Thereafter, the resultant was concentrated under reduced pressure, 7% hydrochloric acid was added thereto, and the mixture was washed with diethyl ether. The pH of the mixture was adjusted to 12 with a 5M potassium hydroxide aqueous solution, extraction was performed with ethyl acetate, and the extract was washed with a saturated sodium chloride aqueous solution. After washing, the washed extract was dried with magnesium sulfate and concentrated under reduced pressure. The concentrated extract was purified through silica gel column chromatography (hexane:ethyl acetate=5:1) to obtain target 4-hydroxy-2,2,6,6-tetraethylpiperidine (1-4) (yield of 2.599 g, percent yield of 60%).

### Synthesis of 4-hydroxy-2,2,6,6-tetraethylpiperidine-1-oxyl (1-5)

In an argon stream, 2.599 g (12.2 mmol) of 4-hydroxy-2,2,6,6-tetraethylpiperidine (1-4) synthesized through the above-described reaction was dissolved in 400 mL of dichloromethane and cooled in an ice bath. 80 mL of a dichloromethane solution containing 24.4 mmol of meta-chloroperoxybenzoic acid (mCPBA) was added dropwise thereto over 45 minutes, and the mixture was stirred at room temperature for 3 hours.

After concentrating the reaction solution under reduced pressure, the obtained crude product was dissolved in diethyl ether. The resultant was washed with a sodium carbonate aqueous solution and then with a saturated sodium chloride aqueous solution, and then dried with magnesium sulfate. The resultant was concentrated under reduced pressure and then purified through silica gel column chromatography (hexane:ethyl acetate=5:1) to obtain target 4-hydroxy-2,2,6,6-tetraethylpiperidine-1-oxyl (1-5) (yield of 1.811 g, percent yield of 65%).

### <Synthesis of Compound (12)>

In an argon stream, 5 mL of dimethylformamide (DMF) was added to 0.349 g (8.00 mmol) of 55% sodium hydride (NaH), and the mixture was stirred at room temperature for 10 minutes. 10 mL of a dimethylformamide solution containing 1.811 g (7.93 mmol) of 4-hydroxy-2,2,6,6-tetraethylpiperidine-1-oxyl (1-5) was added dropwise thereto over 10 minutes, and the mixture was stirred at room temperature for 3 hours.

20 mL of a dimethylformamide solution containing 2.094 g (3.00 mmol) of 2-(3-bromo-2-(bromomethyl)-2-(((1,1,1,3,3,3-hexafluoro-2-(trifluoromethyl)propane-2-synthesized through the above-described reaction was added thereto in an ice bath, and the mixture was stirred at 60° C. for 10 hours.

Water was added to the reaction solution and extraction was performed with ethyl acetate, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (12) (yield of 1.340 g, percent yield of 45%).

When the obtained compound was subjected to mass spectrometry, a peak was confirmed at m/z=992 (M⁺). From this, it was confirmed that the synthesized compound is a compound represented by Formula (12). In addition, the purity of the compound represented by Formula (12) which was conformed through high-performance liquid chromatography (HPLC) was 99.0%. 15 [0093]

### Example 3

### (Synthesis of Compound 13)

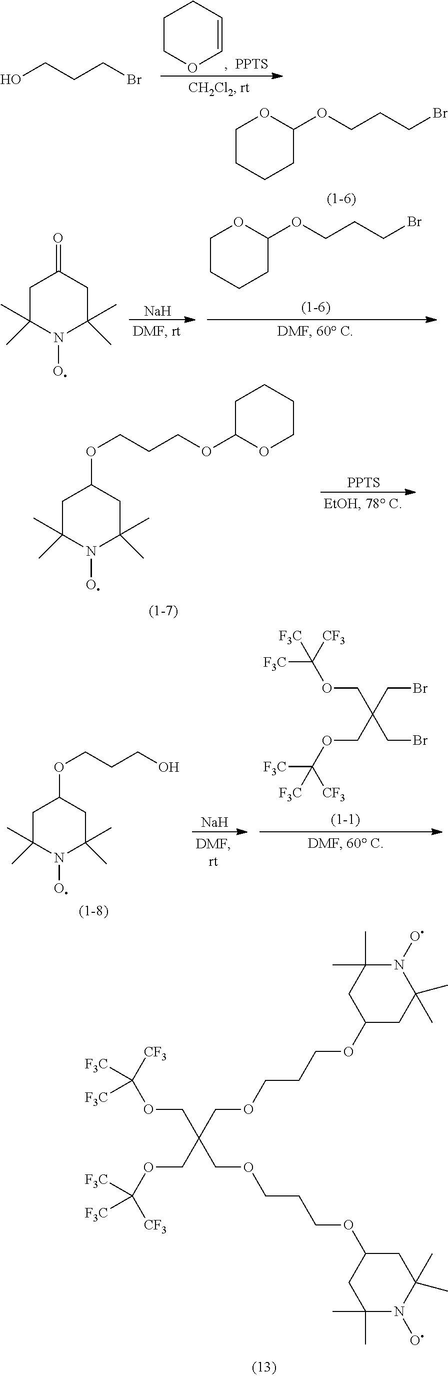

### Synthesis of 2-(2-bromopropoxy)tetrahydro-2H-pyran (1-6)

In an argon stream, 4.170 g (30.0 mmol) of 3-bromo-1-propanol was dissolved in 150 mL of dichloromethane, 3.30 mL (36.0 mmol) of 3,4-dihydro-2H-pyran and 1.508 g (6.00 mmol) of pyridinium p-toluenesulfonate (PPTS) were added thereto in this order, and the mixture was stirred at room temperature for 18 hours.

The reaction solution was concentrated under reduced pressure and then purified through silica gel column chromatography (hexane:ethyl acetate=9:1) to obtain target 2-(2-bromopropoxy)tetrahydro-2H-pyran (1-6) (yield of 5.555 g, percent yield of 83%).

### Synthesis of 2,2,6,6-tetramethyl-4-(2-((tetrahydro-2H-pyran-2-yl)oxy)propoxy)piperidine-1-oxyl (1-7)

In an argon stream, 10 mL of dimethylformamide (DMF) was added to 1.091 g (25.0 mmol) of 55% sodium hydride (NaH), and the mixture was stirred at room temperature for 10 minutes. 20 mL of a dimethylformamide solution containing 3.445 g (20.0 mmol) of 4-hydroxy-2,2,6,6-tetramethylpiperidine-1-oxyl was added dropwise thereto over 10 minutes, and the mixture was stirred at room temperature for 3 hours. 10 mL of a dimethylformamide solution containing 5.555 g (24.9 mmol) of 2-(2-bromopropoxy)tetrahydro-2H-pyran (1-6) synthesized through the above-described reaction was added thereto in an ice bath, and the mixture was stirred at room temperature for 10 hours.

Water was added to the reaction solution and extraction was performed with diethyl ether, and then the extract was washed with water, and the organic layer was dried with magnesium sulfate. The resultant was concentrated under reduced pressure and then purified through silica gel column chromatography (hexane:ethyl acetate=4:1) to obtain target 2,2,6,6-tetramethyl-4-(2-((tetrahydro-2H-pyran-2-yl)oxy)propoxy)piperidine-1-oxyl (1-7) (yield of 5.089 g, percent yield of 65%).

### Synthesis of 4-(3-hydroxypropoxy)-2,2,6,6-tetramethylpiperidine-1-oxyl (1-8)

5.089 g (16.2 mmol) of 2,2,6,6-tetramethyl-4-(2-((tetrahydro-2H-pyran-2-yl)oxy)propoxy)piperidine-1-oxyl (1-7) synthesized through the above-described reaction and 0.407 g (1.62 mmol) of pyridinium p-toluenesulfonate (PPTS) were dissolved in 100 mL of ethanol (EtOH) and stirred at 78° C. for 3 hours.

The reaction solution was concentrated under reduced pressure and then purified through silica gel column chromatography (hexane:ethyl acetate=4:1) to obtain target 4-(3-hydroxypropoxy)-2,2,6,6-tetramethylpiperidine-1-oxyl (1-8) (yield of 3.358 g, percent yield of 90%).

### <Synthesis of Compound (13)>

In an argon stream, 10 mL of dimethylformamide (DMF) was added to 0.655 g (15.0 mmol) of 55% sodium hydride (NaH), and the mixture was stirred at room temperature for 10 minutes. 20 mL of a dimethylformamide solution containing 3.358 g (14.6 mmol) of 4-(3-hydroxypropoxy)-2,2,6,6-tetramethylpiperidine-1-oxyl (1-8) synthesized through the above-described reaction was added dropwise thereto over 10 minutes, and the mixture was stirred at room temperature for 1 hour.

20 mL of a dimethylformamide solution containing 3.839 g (5.50 mmol) of 2-(3-bromo-2-(bromomethyl)-2-(((1,1,1,3,3,3-hexafluoro-2-(trifluoromethyl)propane-2-synthesized through the above-described reaction was added thereto in an ice bath, and the mixture was stirred at 60° C. for 10 hours.

Water was added to the reaction solution and extraction was performed with ethyl acetate, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (13) (yield of 2.193 g, percent yield of 40%).

When the obtained compound was subjected to mass spectrometry, a peak was confirmed at m/z=996 (M⁺). From this, it was confirmed that the synthesized compound is a compound represented by Formula (13). In addition, the purity of the compound represented by Formula (13) which was conformed through high-performance liquid chromatography (HPLC) was 99.0%.

### Example 4

### (Synthesis of Compound 14)

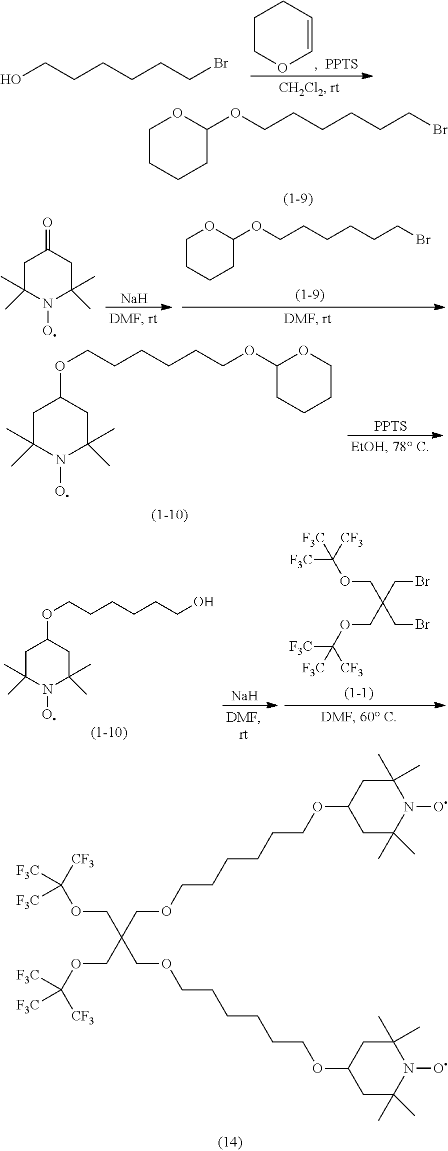

### Synthesis of 2-((6-bromohexyl)oxy)tetrahydro-2H-pyran (1-9)

In an argon stream, 2.72 mL (20.0 mmol) of 6-bromo-1-hexanol was dissolved in 100 mL of dichloromethane, 2.019 g (24.0 mmol) of 3,4-dihydro-2H-pyran and 1.005 g (4.00 mmol) of pyridinium p-toluenesulfonate (PPTS) were added thereto in this order, and the mixture was stirred at room temperature for 18 hours.

The reaction solution was concentrated under reduced pressure and then purified through silica gel column chromatography (hexane:ethyl acetate=9:1) to obtain target 2-((6-bromohexyl)oxy)tetrahydro-2H-pyran (1-9) (yield of 4.667 g, percent yield of 88%).

### Synthesis of 2,2,6,6-tetramethyl-4-(2-(((tetrahydro-2H-pyran-2-yl)oxy)hexyl)oxy)piperidine-1-oxyl (1-10)

In an argon stream, 10 mL of dimethylformamide (DMF) was added to 0.768 g (17.6 mmol) of 55% sodium hydride (NaH), and the mixture was stirred at room temperature for 10 minutes. 20 mL of a dimethylformamide solution containing 2.756 g (16.0 mmol) of 4-hydroxy-2,2,6,6-tetramethylpiperidine-1-oxyl was added dropwise thereto over 10 minutes, and the mixture was stirred at room temperature for 3 hours. 10 mL of a dimethylformamide solution containing 4.667 g (17.6 mmol) of 2-((6-bromohexyl)oxy)tetrahydro-2H-pyran (1-9) synthesized through the above-described reaction was added thereto in an ice bath, and the mixture was stirred at room temperature for 10 hours.

Water was added to the reaction solution and extraction was performed with diethyl ether, and then the extract was washed with water, and the organic layer was dried with magnesium sulfate. The resultant was concentrated under reduced pressure and then purified through silica gel column chromatography (hexane:ethyl acetate=4:1) to obtain target 2,2,6,6-tetramethyl-4-(2-(((tetrahydro-2H-pyran-2-yl)oxy)hexyl)oxy)piperidine-1-oxyl (1-10) (yield of 4.267 g, percent yield of 68%).

### Synthesis of 4-(6-(hydroxyhexyl)oxy)-2,2,6,6-tetramethylpiperidine-1-oxyl (1-11)

4.267 g (11.6 mmol) of 2,2,6,6-tetramethyl-4-(2-(((tetrahydro-2H-pyran-2-yl)oxy)hexyl)oxy)piperidine-1-oxyl (1-10) synthesized through the above-described reaction and 0.292 g (1.16 mmol) of pyridinium p-toluenesulfonate (PPTS) were dissolved in 100 mL of ethanol (EtOH) and stirred at 78° C. for 3 hours.

The reaction solution was concentrated under reduced pressure and then purified through silica gel column chromatography (hexane:ethyl acetate=4:1) to obtain target 4-(6-(hydroxyhexyl)oxy)-2,2,6,6-tetramethylpiperidine-1-oxyl (1-11) (yield of 2.672 g, percent yield of 85%).

### <Synthesis of Compound (14)>

In an argon stream, 5 mL of dimethylformamide (DMF) was added to 0.436 g (10.0 mmol) of 55% sodium hydride (NaH), and the mixture was stirred at room temperature for 10 minutes. 10 mL of a dimethylformamide solution containing 2.672 g (9.81 mmol) of 4-(6-(hydroxyhexyl)oxy)-2,2,6,6-tetramethylpiperidine-1-oxyl (1-11) synthesized through the above-described reaction was added dropwise thereto over 10 minutes, and the mixture was stirred at room temperature for 1 hour.

20 mL of a dimethylformamide solution containing 2.652 g (3.80 mmol) of 2-(3-bromo-2-(bromomethyl)-2-(((1,1,1,3,3,3-hexafluoro-2-(trifluoromethyl)propane-2-synthesized through the above-described reaction was added thereto in an ice bath, and the mixture was stirred at 60° C. for 10 hours.

Water was added to the reaction solution and extraction was performed with ethyl acetate, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (14) (yield of 1.602 g, percent yield of 39%).

When the obtained compound was subjected to mass spectrometry, a peak was confirmed at m/z=1080 (M⁺). From this, it was confirmed that the synthesized compound is a compound represented by Formula (14). In addition, the purity of the compound represented by Formula (14) which was conformed through high-performance liquid chromatography (HPLC) was 98.5%.

### Example 5

### (Synthesis of Compound 15)

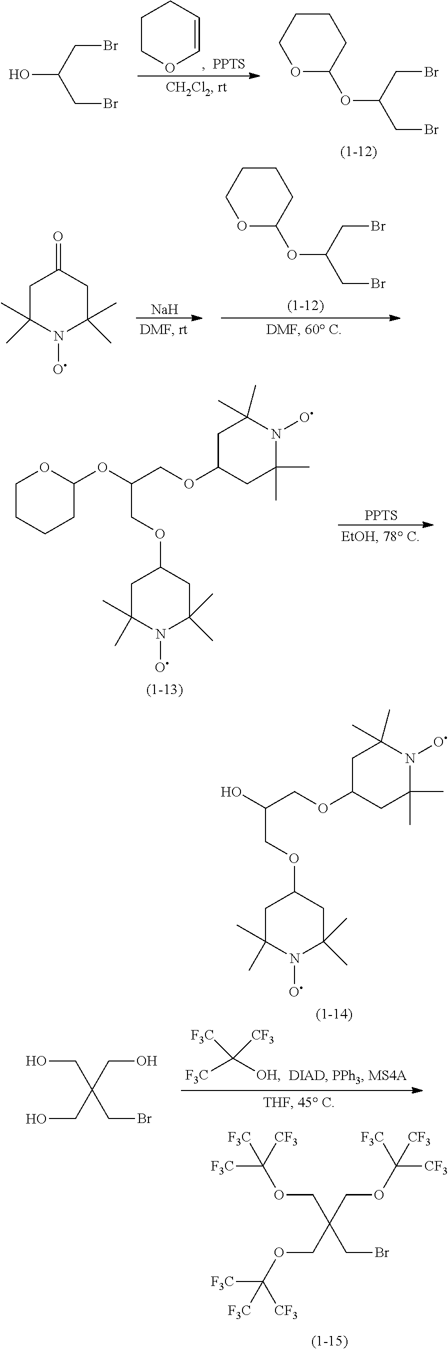

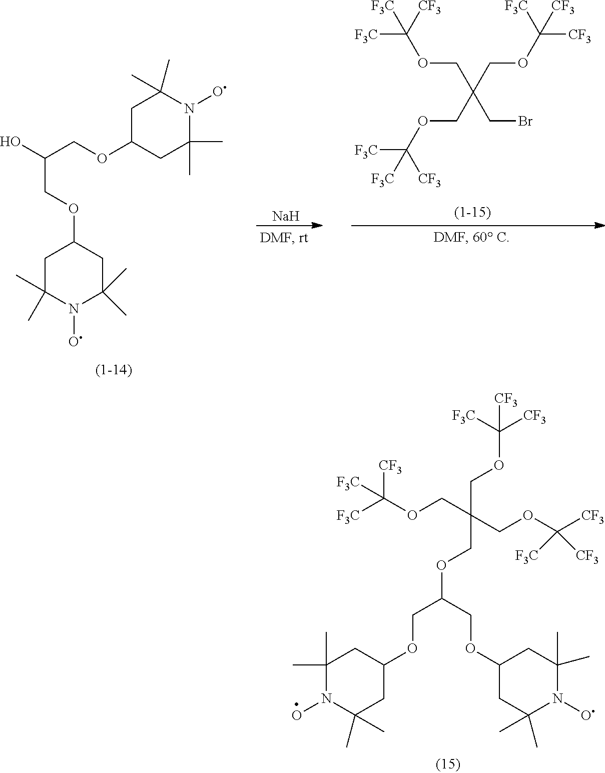

### Synthesis of 2-((1,3-dibromopropane-2-yl)oxy)tetrahydro-2H-pyran (1-12)

In an argon stream, 4.358 g (20.0 mmol) of 1,3-dibromo-2-propanol was dissolved in 100 mL of dichloromethane, 0.681 g (24.0 mmol) of 3,4-dihydro-2H-pyran and 1.001 g (4.00 mmol) of pyridinium p-toluenesulfonate (PPTS) were added thereto in this order, and the mixture was stirred at room temperature for 18 hours.

The reaction solution was concentrated under reduced pressure and then purified through silica gel column chromatography (hexane:ethyl acetate=9:1) to obtain 2-((1,3-dibromopropane-2-yl)oxy)tetrahydro-2H-pyran (1-12) (yield of 4.832 g, percent yield of 80%).

### <Synthesis of Compound (1-13)>

In an argon stream, 10 mL of dimethylformamide (DMF) was added to 1.135 g (26.0 mmol) of 55% sodium hydride (NaH), and the mixture was stirred at room temperature for 10 minutes. 20 mL of a dimethylformamide solution containing 4.479 g (26.0 mmol) of 4-hydroxy-2,2,6,6-tetramethylpiperidine-1-oxyl was added dropwise thereto over 10 minutes, and the mixture was stirred at room temperature for 3 hours. 20 mL of a dimethylformamide solution containing 3.020 g (10.0 mmol) of 2-((1,3-dibromopropane-2-yl)oxy)tetrahydro-2H-pyran (1-12) synthesized through the above-described reaction was added thereto in an ice bath, and the mixture was stirred at 60° C. for 10 hours.

Water was added to the reaction solution and extraction was performed with diethyl ether, and then the extract was washed with water, and the organic layer was dried with magnesium sulfate. The dried organic layer was concentrated under reduced pressure and then purified through silica gel column chromatography (hexane:ethyl acetate=4:1) to obtain a target compound (1-13) (yield of 1.454 g, percent yield of 30%).

### <Synthesis of Compound (1-14)>

1.454 g (3.00 mmol) of the compound (1-13) synthesized through the above-described reaction and 0.075 g (0.30 mmol) of pyridinium p-toluenesulfonate (PPTS) were dissolved in 100 mL of ethanol (EtOH) and stirred at 78° C. for 3 hours.

The reaction solution was concentrated under reduced pressure and then purified through silica gel column chromatography (hexane:ethyl acetate=4:1) to obtain a target compound (1-14) (yield of 1.021 g, percent yield of 85%).

### Synthesis of 2-(3-bromo-2,2,-bis(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-15)

In an argon stream, 5.000 g (25.1 mmol) of 2-(bromomethyl)-2-(hydroxymethyl)-1,3-propanediol, 27.777 g (105.9 mmol) of triphenylphosphine (PPh₃), 10.000 g of molecular sieves (MS) 4 A, and 130 mL of tetrahydrofuran (THF) were mixed with each other and cooled in an ice bath. 20.6 mL (105.9 mmol) of diisopropyl azodicarboxylate (DIAD) was added dropwise thereto over 10 minutes, and the mixture was stirred for 20 minutes. 25.000 g (105.9 mmol) of nonafluoro-tert-butanol was added thereto at one time, and the mixture was stirred at 45° C. for 72 hours.

After the reaction solution was filtered, it was concentrated under reduced pressure and purified through silica gel column chromatography (hexane:ethyl acetate=9:1) to obtain a target compound 2-(3-bromo-2,2,-bis(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-15) (yield of 16.055 g, percent yield of 75%).

### <Synthesis of Compound (15)>

In an argon stream, 5 mL of dimethylformamide (DMF) was added to 0.123 g (2.81 mmol) of 55% sodium hydride (NaH), and the mixture was stirred at room temperature for 10 minutes. 10 mL of a dimethylformamide solution containing 1.021 g (2.55 mmol) of the compound (1-14) synthesized through the above-described reaction was added dropwise thereto over 10 minutes, and the mixture was stirred at room temperature for 1 hour.

30 mL of a dimethylformamide solution containing 2.730 g (3.32 mmol) of 2-(3-bromo-2,2,-bis(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-15) synthesized through the above-described reaction was added thereto in an ice bath, and the mixture was stirred at 60° C. for 10 hours.

Water was added to the reaction solution and extraction was performed with ethyl acetate, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (15) (yield of 1.346 g, percent yield of 45%).

When the obtained compound was subjected to mass spectrometry, a peak was confirmed at m/z=1172 (M⁺). From this, it was confirmed that the synthesized compound is a compound represented by Formula (15). In addition, the purity of the compound represented by Formula (15) which was conformed through high-performance liquid chromatography (HPLC) was 99.5%. 20 [0128]

### Example 6

### (Synthesis of Compound 16)

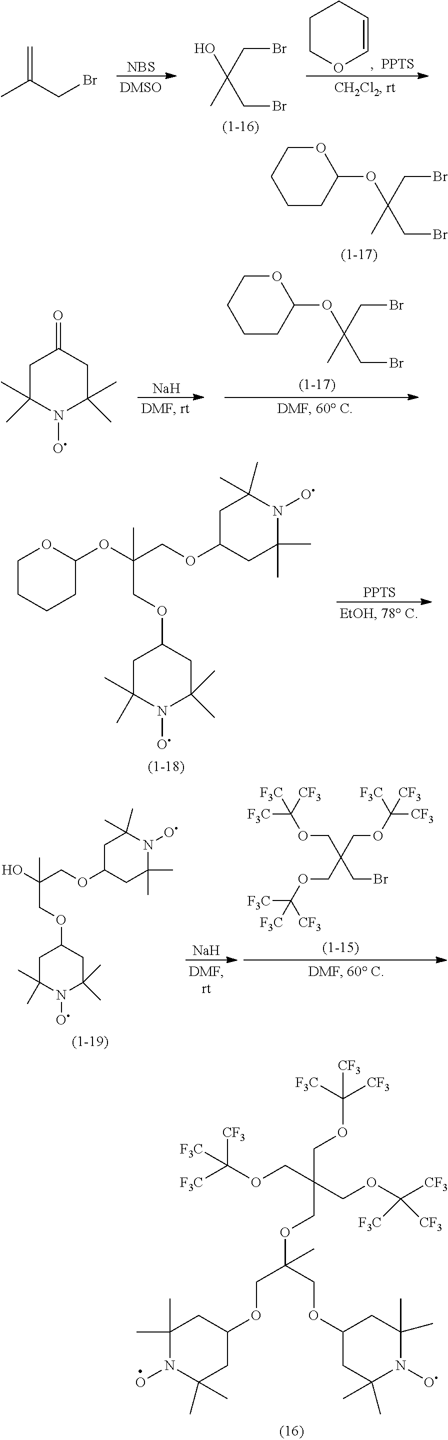

### Synthesis of 1,3-dibromo-2-methyl-2-propanol (1-16)

In an argon stream, 2.000 g (15.0 mmol) of 3-bromo-2-methyl-1-propene was dissolved in 90 mL of dimethyl sulfoxide (DMSO) and 5 mL of water and cooled to −10° C. 5.270 g (30.0 mmol) of N-bromosuccinimide (NBS) was added thereto little by little over 15 minutes, and the mixture was stirred for 3 hours.

Water was added to the reaction solution and extraction was performed with diethyl ether, and the organic layer was washed with a saturated sodium chloride aqueous solution and then dried with magnesium sulfate. The organic layer was concentrated under reduced pressure and then distilled under reduced pressure to obtain target 1,3-dibromo-2-methyl-2-propanol (1-16) (yield of 1.565 g, percent yield of 45%).

### Synthesis of 2-((1,3-dibromo-2-methylpropane-2-yl)oxy)tetrahydro-2H-pyran (1-17)

In an argon stream, 1.565 g (6.75 mmol) of 1,3-dibromo-2-methyl-2-propanol (1-16) synthesized through the above-described reaction was dissolved in 100 mL of dichloromethane, 0.681 g (8.10 mmol) of 3,4-dihydro-2H-pyran and 0.339 g (1.35 mmol) of pyridinium p-toluenesulfonate (PPTS) were added thereto in this order, and the mixture was stirred at room temperature for 18 hours.

The reaction solution was concentrated under reduced pressure and then purified through silica gel column chromatography (hexane:ethyl acetate=9:1) to obtain 2-((1,3-dibromo-2-methylpropane-2-yl)oxy)tetrahydro-2H-pyran (1-17) (yield of 1.707 g, percent yield of 80%).

### <Synthesis of Compound (1-18)>

In an argon stream, 10 mL of dimethylformamide (DMF) was added to 0.611 g (14.0 mmol) of 55% sodium hydride (NaH), and the mixture was stirred at room temperature for 10 minutes. 20 mL of a dimethylfomamide solution containing 2.412 g (14.0 mmol) of 4-hydroxy-2,2,6,6-tetramethylpiperidine-1-oxyl was added dropwise thereto over 10 minutes, and the mixture was stirred at room temperature for 3 hours. 20 mL of a dimethylformamide solution containing 1.707 g (5.40 mmol) of 2-((1,3-dibromo-2-methylpropane-2-yl)oxy)tetrahydro-2H-pyran (1-17) synthesized through the above-described reaction was added thereto in an ice bath, and the mixture was stirred at 60° C. for 12 hours.

Water was added to the reaction solution and extraction was performed with diethyl ether, and then the extract was washed with water, and the organic layer was dried with magnesium sulfate. The dried organic layer was concentrated under reduced pressure and then purified through silica gel column chromatography (hexane:ethyl acetate=4:1) to obtain a target compound (1-18) (yield of 1.885 g, percent yield of 70%).

### <Synthesis of Compound (1-19)>

1.885 g (3.78 mmol) of the compound (1-18) synthesized through the above-described reaction and 0.095 g (0.38 mmol) of pyridinium p-toluenesulfonate (PPTS) were dissolved in 100 mL of ethanol (EtOH) and stirred at 78° C. for 3 hours.

The reaction solution was concentrated under reduced pressure and then purified through silica gel column chromatography (hexane:ethyl acetate=4:1) to obtain a target compound (1-19) (yield of 1.332 g, percent yield of 85%).

### <Synthesis of Compound (16)>

In an argon stream, 5 mL of dimethylformamide (DMF) was added to 0.154 g (3.53 mmol) of 55% sodium hydride (NaH), and the mixture was stirred at room temperature for 10 minutes. 10 mL of a dimethylformamide solution containing 1.332 g (3.21 mmol) of the compound (1-19) synthesized through the above-described reaction was added dropwise thereto over 10 minutes, and the mixture was stirred at room temperature for 1 hour.

30 mL of a dimethylformamide solution containing 3.560 g (4.17 mmol) of 2-(3-bromo-2,2,-bis(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-15) synthesized through the above-described reaction was added thereto in an ice bath, and the mixture was stirred at 60° C. for 10 hours.

Water was added to the reaction solution and extraction was performed with ethyl acetate, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (16) (yield of 1.905 g, percent yield of 50%).

When the obtained compound was subjected to mass spectrometry, a peak was confirmed at m/z=1186 (M⁺). From this, it was confirmed that the synthesized compound is a compound represented by Formula (16). In addition, the purity of the compound represented by Formula (16) which was conformed through high-performance liquid chromatography (HPLC) was 99.1%.

### Example 7

### (Synthesis of Compound 17)

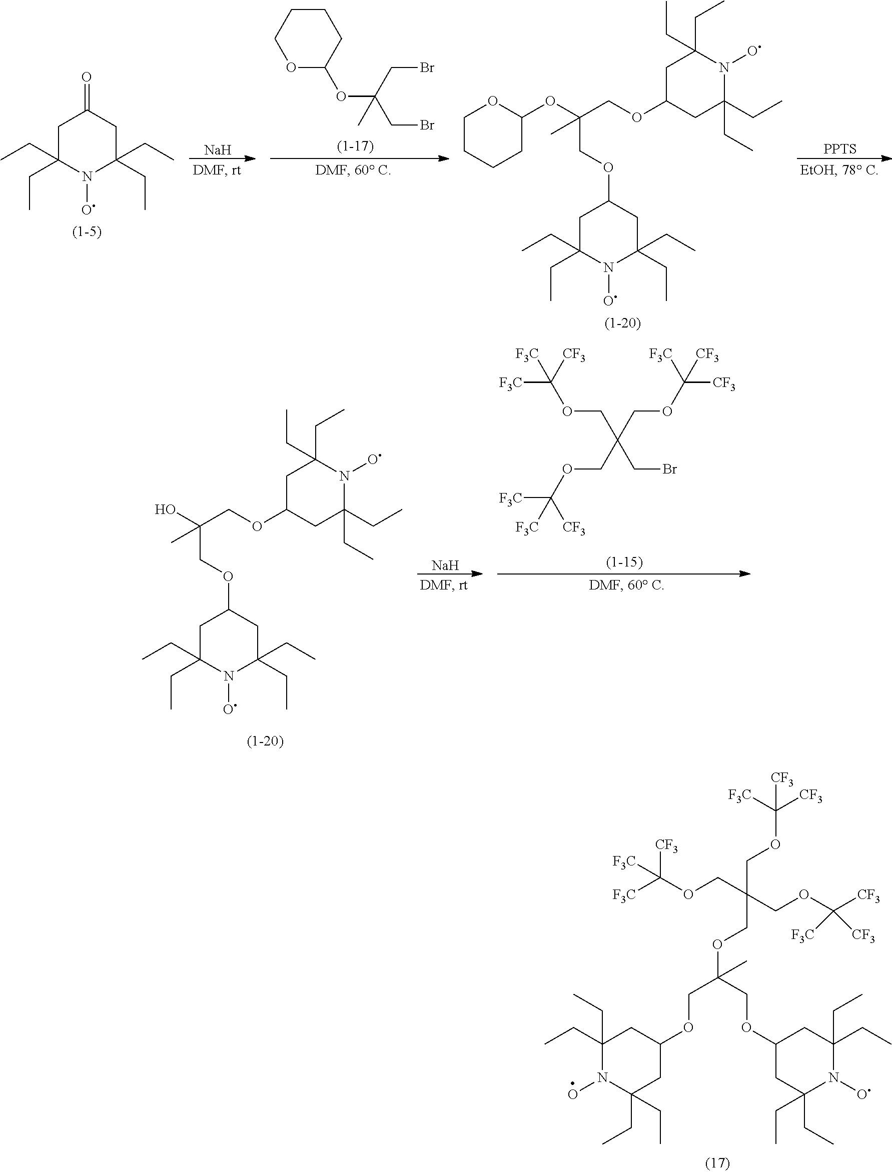

### <Synthesis of Compound (1-20)>

In an argon stream, 5 mL of dimethylformamide (DMF) was added to 0.384 g (8.80 mmol) of 55% sodium hydride (NaH), and the mixture was stirred at room temperature for 10 minutes. 10 mL of a dimethylformamide solution containing 1.826 g (8.00 mmol) of 4-hydroxy-2,2,6,6-tetraethylpiperidine-1-oxyl (1-5) synthesized through the above-described reaction was added dropwise thereto over 10 minutes, and the mixture was stirred at room temperature for 3 hours. 10 mL of a dimethylformamide solution containing 1.052 g (3.33 mmol) of 2-((1,3-dibromo-2-methylpropane-2-yl)oxy)tetrahydro-2H-pyran (1-17) synthesized through the above-described reaction was added thereto in an ice bath, and the mixture was stirred at 60° C. for 10 hours.

Water was added to the reaction solution and extraction was performed with diethyl ether, and then the extract was washed with water, and the organic layer was dried with magnesium sulfate. The dried organic layer was concentrated under reduced pressure and then purified through silica gel column chromatography (hexane:ethyl acetate=4:1) to obtain a target compound (1-20) (yield of 1.383 g, percent yield of 68%).

### <Synthesis of Compound (1-21)>

1.383 g (2.26 mmol) of the compound (1-20) synthesized through the above-described reaction and 0.058 g (0.23 mmol) of pyridinium p-toluenesulfonate (PPTS) were dissolved in 100 mL of ethanol (EtOH) and stirred at 78° C. for 3 hours.

The reaction solution was concentrated under reduced pressure and then purified through silica gel column chromatography (hexane:ethyl acetate=4:1) to obtain a target compound (1-21) (yield of 1.048 g, percent yield of 88%).

### <Synthesis of Compound (17)>

In an argon stream, 5 mL of dimethylformamide (DMF) was added to 0.087 g (2.00 mmol) of 55% sodium hydride (NaH), and the mixture was stirred at room temperature for 10 minutes. 10 mL of a dimethylformamide solution containing 1.048 g (1.99 mmol) of the compound (1-21) synthesized through the above-described reaction was added dropwise thereto over 10 minutes, and the mixture was stirred at room temperature for 1 hour.

20 mL of a dimethylformamide solution containing 2.218 g (2.60 mmol) of 2-(3-bromo-2,2,-bis(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-15) synthesized through the above-described reaction was added thereto in an ice bath, and the mixture was stirred at 60° C. for 15 hours.

Water was added to the reaction solution and extraction was performed with ethyl acetate, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (17) (yield of 1.525 g, percent yield of 59%).

When the obtained compound was subjected to mass spectrometry, a peak was confirmed at m/z=1299 (M⁺). From this, it was confirmed that the synthesized compound is a compound represented by Formula (17). In addition, the purity of the compound represented by Formula (17) which was conformed through high-performance liquid chromatography (HPLC) was 98.8%.

### Example 8

### (Synthesis of Compound 18)

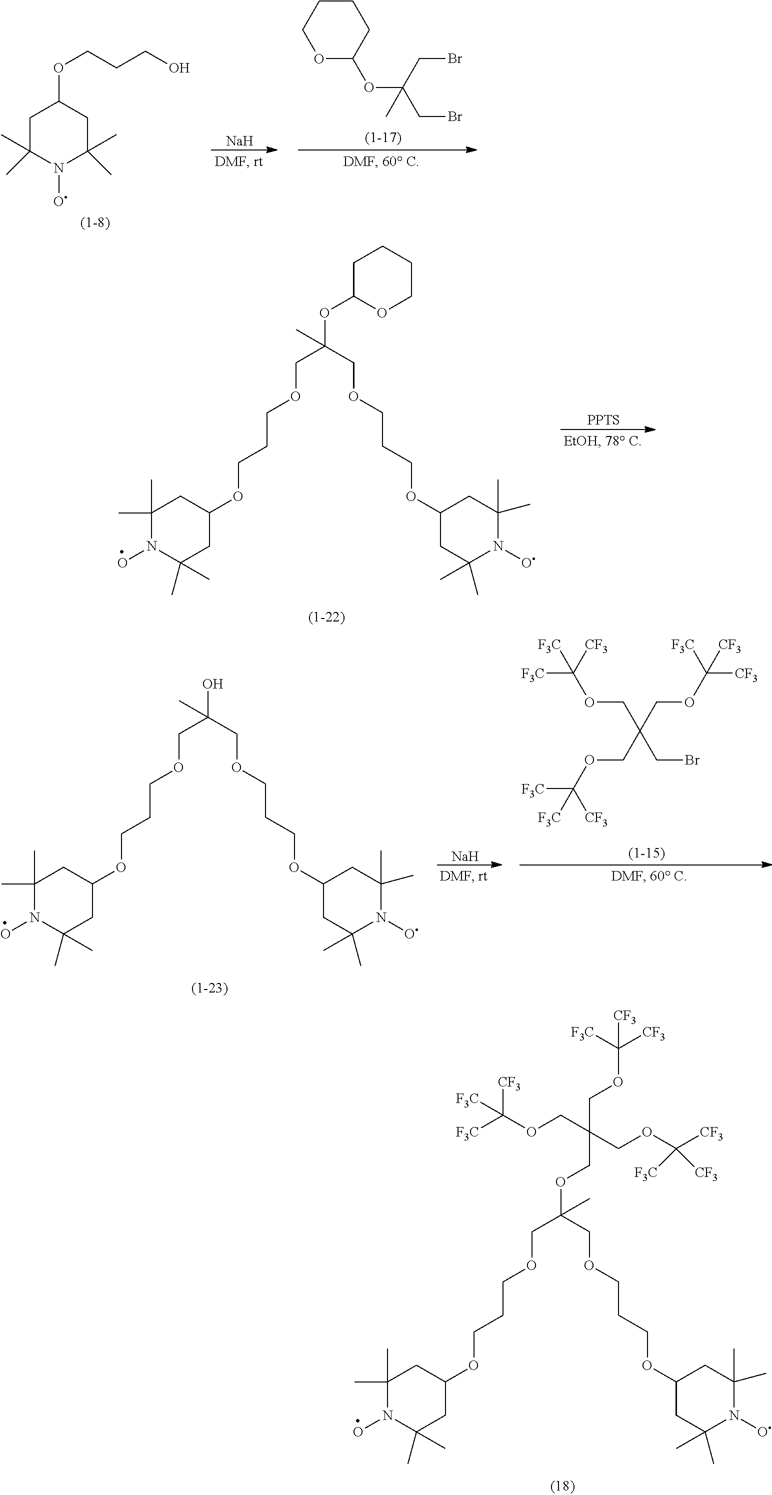

### <Synthesis of Compound (1-22)>

In an argon stream, 5 mL of dimethylformamide (DMF) was added to 0.480 g (11.0 mmol) of 55% sodium hydride (NaH), and the mixture was stirred at room temperature for 10 minutes. 10 mL of a dimethylformamide solution containing 2.303 g (10.0 mmol) of 4-(3-hydroxypropoxy)-2,2,6,6-tetramethylpiperidine-1-oxyl (1-8) synthesized through the above-described reaction was added dropwise thereto over 10 minutes, and the mixture was stirred at room temperature for 1 hour. 10 mL of a dimethylformamide solution containing 1.318 g (4.17 mmol) of 2-((1,3-dibromo-2-methylpropane-2-yl)oxy)tetrahydro-2H-pyran (1-17) synthesized through the above-described reaction was added thereto in an ice bath, and the mixture was stirred at 60° C. for 10 hours.

Water was added to the reaction solution and extraction was performed with diethyl ether, and then the extract was washed with water, and the organic layer was dried with magnesium sulfate. The dried organic layer was concentrated under reduced pressure and then purified through silica gel column chromatography (hexane:ethyl acetate=4:1) to obtain a target compound (1-22) (yield of 1.333 g, percent yield of 52%).

<Synthesis of compound (1-23)>

1.333 g (2.17 mmol) of the compound (1-22) synthesized through the above-described reaction and 0.055 g (0.22 mmol) of pyridinium p-toluenesulfonate (PPTS) were dissolved in 100 mL of ethanol (EtOH) and stirred at 78° C. for 3 hours.

The reaction solution was concentrated under reduced pressure and then purified through silica gel column chromatography (hexane:ethyl acetate=4:1) to obtain a target compound (1-23) (yield of 1.037 g, percent yield of 90%).

<Synthesis of compound (18)>

In an argon stream, 5 mL of dimethylformamide (DMF) was added to 0.087 g (2.00 mmol) of 55% sodium hydride (NaH), and the mixture was stirred at room temperature for 10 minutes. 20 mL of a dimethylformamide solution containing 1.037 g (1.95 mmol) of the compound (1-23) synthesized through the above-described reaction was added dropwise thereto over 10 minutes, and the mixture was stirred at room temperature for 1 hour. 20 mL of a dimethylformamide solution containing 2.218 g (2.60 mmol) of 2-(3-bromo-2,2,-bis(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-15) synthesized through the above-described reaction was added thereto in an ice bath, and the mixture was stirred at 60° C. for 13 hours.

Water was added to the reaction solution and extraction was performed with ethyl acetate, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (18) (yield of 1.093 g, percent yield of 43%).

When the obtained compound was subjected to mass spectrometry, a peak was confirmed at m/z=1302 (M⁺). From this, it was confirmed that the synthesized compound is a compound represented by Formula (18). In addition, the purity of the compound represented by Formula (18) which was conformed through high-performance liquid chromatography (HPLC) was 98.8%.

### Example 9

### (Synthesis of Compound 19)

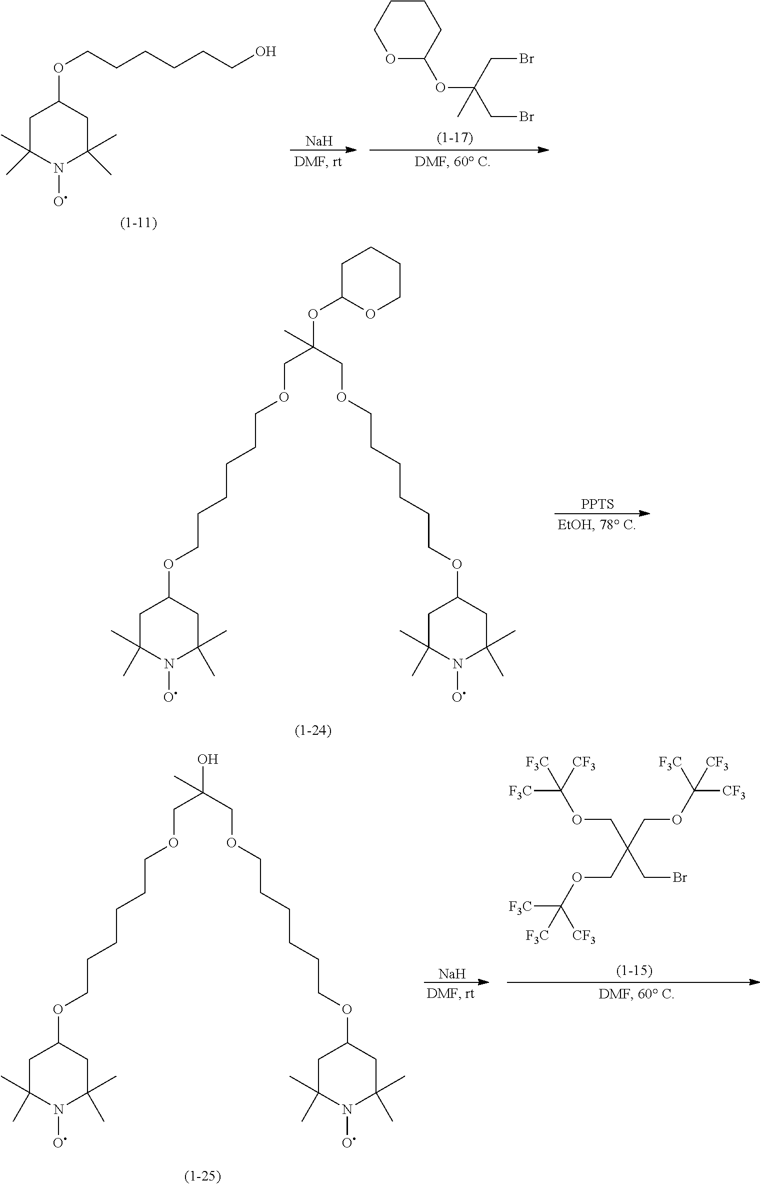

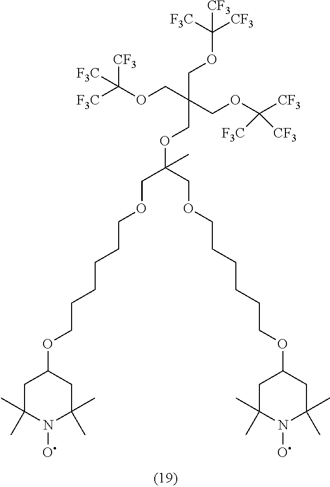

### <Synthesis of Compound (1-24)>

In an argon stream, 5 mL of dimethylformamide (DMF) was added to 0.480 g (11.0 mmol) of 55% sodium hydride (NaH), and the mixture was stirred at room temperature for 10 minutes. 10 mL of a dimethylformamide solution containing 2.724 g (10.0 mmol) of 4-(6-(hydroxyhexyl)oxy)-2,2,6,6-tetramethylpiperidine-1-oxyl (1-11) synthesized through the above-described reaction was added dropwise thereto over 10 minutes, and the mixture was stirred at room temperature for 1 hour. 10 mL of a dimethylformamide solution containing 1.318 g (4.17 mmol) of 2-((1,3-dibromo-2-methylpropane-2-yl)oxy)tetrahydro-2H-pyran (1-17) synthesized through the above-described reaction was added thereto in an ice bath, and the mixture was stirred at 60° C. for 10 hours.

Water was added to the reaction solution and extraction was performed with diethyl ether, and then the extract was washed with water, and the organic layer was dried with magnesium sulfate. The dried organic layer was concentrated under reduced pressure and then purified through silica gel column chromatography (hexane:ethyl acetate=4:1) to obtain a target compound (1-24) (yield of 1.399 g, percent yield of 48%).

### <Synthesis of Compound (1-25)>

1.399 g (2.00 mmol) of the compound (1-24) synthesized through the above-described reaction and 0.050 g (0.20 mmol) of pyridinium p-toluenesulfonate (PPTS) were dissolved in 100 mL of ethanol (EtOH) and stirred at 78° C. for 3 hours.

The reaction solution was concentrated under reduced pressure and then purified through silica gel column chromatography (hexane:ethyl acetate=4:1) to obtain a target compound (1-25)) (yield of 1.008 g, percent yield of 82%).

### <Synthesis of Compound (19)>

In an argon stream, 5 mL of dimethylformamide (DMF) was added to 0.078 g (1.80 mmol) of 55% sodium hydride (NaH), and the mixture was stirred at room temperature for 10 minutes. 20 mL of a dimethylformamide solution containing 1.008 g (1.64 mmol) of the compound (1-25) synthesized through the above-described reaction was added dropwise thereto over 10 minutes, and the mixture was stirred at room temperature for 3 hours. 20 mL of a dimethylformamide solution containing 1.819 g (2.13 mmol) of 2-(3-bromo-2,2,-bis(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-15) synthesized through the above-described reaction was added thereto in an ice bath, and the mixture was stirred at 60° C. for 16 hours.

Water was added to the reaction solution and extraction was performed with ethyl acetate, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (19) (yield of 1.024 g, percent yield of 45%).

When the obtained compound was subjected to mass spectrometry, a peak was confirmed at m/z=1387 (M⁺). From this, it was confirmed that the synthesized compound is a compound represented by Formula (19). In addition, the purity of the compound represented by Formula (19) which was conformed through high-performance liquid chromatography (HPLC) was 98.3%.

### Example 10

### (Synthesis of Compound 20)

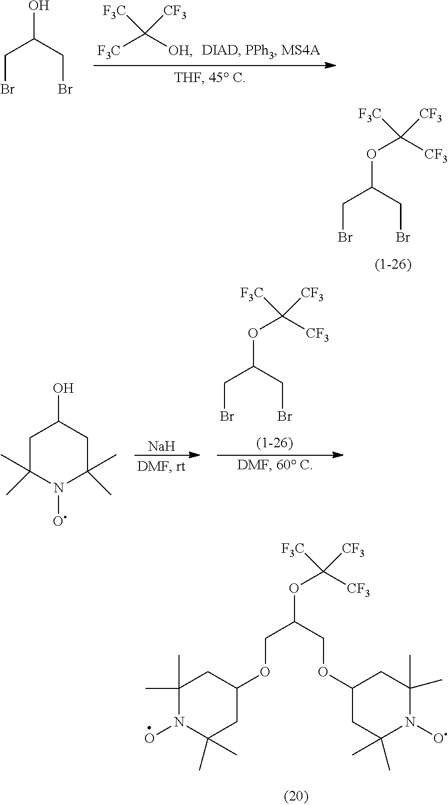

### Synthesis of 2-((1,3-dibromopropane-2-yl)oxy)propoxy)-1,1,1,3,3,3-hexafluoro-2-(trifluoromethyl)propane (1-26)

In an argon stream, 6.537 g (30.0 mmol) of 1,3-dibromo-2-propanol, 10.229 g (39.0 mmol) of triphenylphosphine (PPh₃), 5.000 g of molecular sieves (MS) 4 A, and 100 mL of tetrahydrofuran (THF) were mixed with each other and cooled in an ice bath. 7.68 mL (39.0 mmol) of diisopropyl azodicarboxylate (DIAD) was added dropwise thereto over 10 minutes, and the mixture was stirred for 20 minutes. 9.206 g (39.0 mmol) of nonafluoro-tert-butanol was added thereto at one time, and the mixture was stirred at 45° C. for 24 hours.

After the reaction solution was filtered, it was concentrated under reduced pressure and purified through silica gel column chromatography (hexane:ethyl acetate=95:5) to obtain a target compound 2-((1,3-dibromopropane-2-yl)oxy)propoxy)-1,1,1,3,3,3-hexafluoro-2-(trifluoromethyl)propane (1-26) (yield of 10.462 g, percent yield of 80%).

### <Synthesis of Compound (20)>

In an argon stream, 10 mL of dimethylformamide (DMF) was added to 0.611 g (14.0 mmol) of 55% sodium hydride (NaH), and the mixture was stirred at room temperature for 10 minutes. 20 mL of a dimethylformamide solution containing 2.239 g (13.0 mmol) of 4-hydroxy-2,2,6,6-tetramethylpiperidine-1-oxyl was added dropwise thereto over 10 minutes, and the mixture was stirred at room temperature for 3 hours. 20 mL of a dimethylformamide solution containing 2.180 g (5.00 mmol) of 2-((1,3-dibromopropane-2-yl)oxy)propoxy)-1,1,1,3,3,3-hexafluoro-2-(trifluoromethyl)propane (1-26) synthesized through the above-described reaction was added thereto in an ice bath, and the mixture was stirred at 60° C. for 12 hours.

Water was added to the reaction solution and extraction was performed with diethyl ether, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (20) (yield of 2.010 g, percent yield of 65%).

When the obtained compound was subjected to mass spectrometry, a peak was confirmed at m/z=618 (M⁺). From this, it was confirmed that the synthesized compound is a compound represented by Formula (20). In addition, the purity of the compound represented by Formula (20) which was conformed through high-performance liquid chromatography (HPLC) was 99.6%.

### Example 11

### (Synthesis of Compound 21)

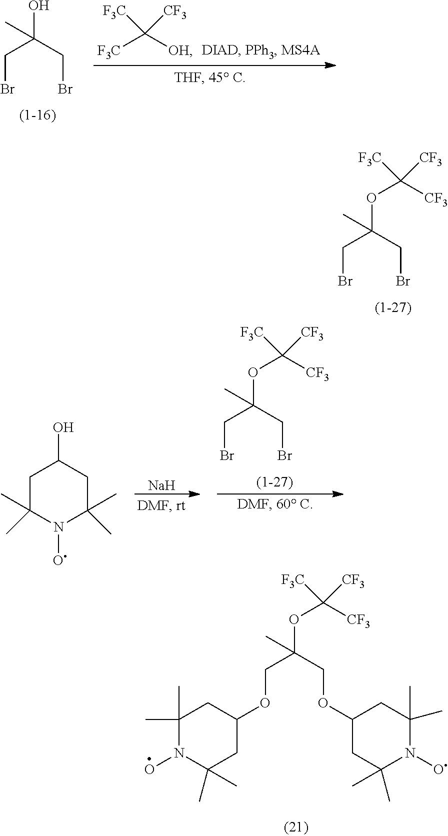

### Synthesis of 2-((1,3-dibromo-2-methylpropane-2-yl)oxy)propoxy)-1,1,1,3,3,3-hexafluoro-2-(trifluoromethyl)propane (1-27)

In an argon stream, 1.160 g (5.00 mmol) of 1,3-dibromo-2-methyl-2-propanol (1-16) synthesized through the above-described reaction, 1.705 g (6.50 mmol) of triphenylphosphine (PPh₃), 3.000 g of molecular sieves (MS) 4 A, and 50 mL of tetrahydrofuran (THF) were mixed with each other and cooled in an ice bath. 1.28 mL (6.50 mmol) of diisopropyl azodicarboxylate (DIAD) was added dropwise thereto over 5 minutes, and the mixture was stirred for 20 minutes. 1.534 g (6.50 mmol) of nonafluoro-tert-butanol was added thereto at one time, and the mixture was stirred at 45° C. for 24 hours.

After the reaction solution was filtered, it was concentrated under reduced pressure and purified through silica gel column chromatography (hexane:ethyl acetate=95:5) to obtain a target compound 2-((1,3-dibromo-2-methylpropane-2-yl)oxy)propoxy)-1,1,1,3,3,3-hexafluoro-2-(trifluoromethyl)propane (1-27) (yield of 1.732 g, percent yield of 77%).

### <Synthesis of Compound (21)>

In an argon stream, 10 mL of dimethylformamide (DMF) was added to 0.480 g (11.0 mmol) of 55% sodium hydride (NaH), and the mixture was stirred at room temperature for 10 minutes. 20 mL of a dimethylformamide solution containing 1.723 g (10.0 mmol) of 4-hydroxy-2,2,6,6-tetramethylpiperidine-1-oxyl was added dropwise thereto over 10 minutes, and the mixture was stirred at room temperature for 3 hours. 20 mL of a dimethylformamide solution containing 1.732 g (3.85 mmol) of 2-((1,3-dibromo-2-methylpropane-2-yl)oxy)propoxy)-1,1,1,3,3,3-hexafluoro-2-(trifluoromethyl)propane (1-27) synthesized through the above-described reaction was added thereto in an ice bath, and the mixture was stirred at 60° C. for 12 hours.

Water was added to the reaction solution and extraction was performed with diethyl ether, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (21) (yield of 1.340 g, percent yield of 55%).

When the obtained compound was subjected to mass spectrometry, a peak was confirmed at m/z=632 (M⁺). From this, it was confirmed that the synthesized compound is a compound represented by Formula (21). In addition, the purity of the compound represented by Formula (21) which was conformed through high-performance liquid chromatography (HPLC) was 99.0%.

### Example 12

### (Synthesis of Compound 22)

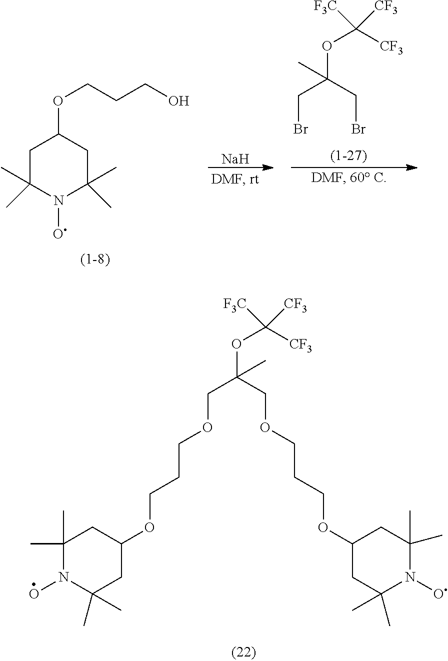

### <Synthesis of Compound (22)>

In an argon stream, 10 mL of dimethylformamide (DMF) was added to 0.480 g (11.0 mmol) of 55% sodium hydride (NaH), and the mixture was stirred at room temperature for 10 minutes. 20 mL of a dimethylformamide solution containing 2.303 g (10.0 mmol) of 4-(3-hydroxypropoxy)-2,2,6,6-tetramethylpiperidine-1-oxyl (1-8) synthesized through the above-described reaction was added dropwise thereto over 10 minutes, and the mixture was stirred at room temperature for 3 hours. 20 mL of a dimethylformamide solution containing 1.710 g (3.80 mmol) of 2-((1,3-dibromo-2-methylpropane-2-yl)oxy)propoxy)-1,1,1,3,3,3-hexafluoro-2-(trifluoromethyl)propane (1-27) synthesized through the above-described reaction was added thereto in an ice bath, and the mixture was stirred at 60° C. for 12 hours.

Water was added to the reaction solution and extraction was performed with diethyl ether, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (22) (yield of 1.366 g, percent yield of 48%).

When the obtained compound was subjected to mass spectrometry, a peak was confirmed at m/z=748 (M⁺). From this, it was confirmed that the synthesized compound is a compound represented by Formula (22). In addition, the purity of the compound represented by Formula (22) which was conformed through high-performance liquid chromatography (HPLC) was 98.2%.

### Example 13

### (Synthesis of Compound 23)

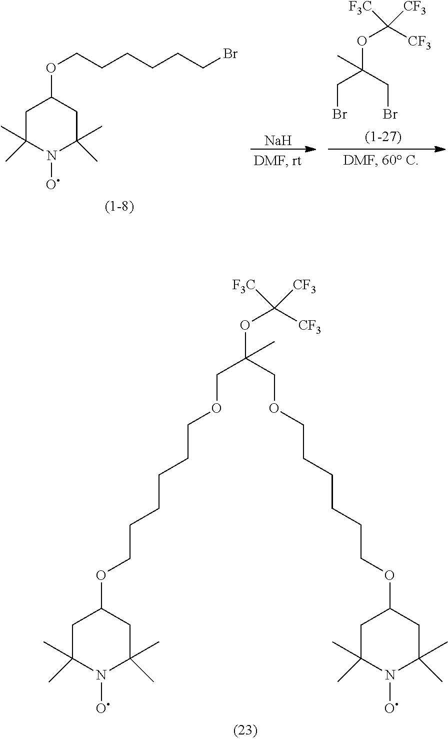

### <Synthesis of Compound (23)>

In an argon stream, 10 mL of dimethylformamide (DMF) was added to 0.480 g (11.0 mmol) of 55% sodium hydride (NaH), and the mixture was stirred at room temperature for 10 minutes. 20 mL of a dimethylformamide solution containing 2.724 g (10.0 mmol) of 4-(6-(hydroxyhexyl)oxy)-2,2,6,6-tetramethylpiperidine-1-oxyl (1-11) synthesized through the above-described reaction was added dropwise thereto over 10 minutes, and the mixture was stirred at room temperature for 3 hours. 20 mL of a dimethylformamide solution containing 1.710 g (3.80 mmol) of 2-((1,3-dibromo-2-methylpropane-2-yl)oxy)propoxy)-1,1,1,3,3,3-hexafluoro-2-(trifluoromethyl)propane (1-27) synthesized through the above-described reaction was added thereto in an ice bath, and the mixture was stirred at 60° C. for 15 hours.

Water was added to the reaction solution and extraction was performed with diethyl ether, and then the extract was washed with water, and the organic layer was dried with magnesium sulfate. The dried organic layer was concentrated under reduced pressure and then purified through silica gel column chromatography (hexane:ethyl acetate=9:1) to obtain a target compound (23) (yield of 1.266 g, percent yield of 40%).

When the obtained compound was subjected to mass spectrometry, a peak was confirmed at m/z=832 (M⁺). From this, it was confirmed that the synthesized compound is a compound represented by Formula (23). In addition, the purity of the compound represented by Formula (23) which was conformed through high-performance liquid chromatography (HPLC) was 98.2%.

### Comparative Example 1

1,3-bis(2,2,2-trifluoro-1,1-bis(trifluoromethyl(ethoxy)-2,2-bis((2,2,2-trifluoro)-1,1-bis(trifluoromethyl)ethoxy)methyl)propane (PERFECTA, manufactured by Aldrich) represented by Formula (A1) below was prepared.

### Comparative Example 2

Trifluoromethylbenzene represented by Formula (A2) and 4-hydroxy-2,2,6,6-tetramethylpiperidine-1-oxyl free radical represented by Formula (A3) below (TEMPOL, manufactured by Tokyo Chemical Industry Co., Ltd.) were mixed with each other at a ratio ((A2):(A3)) of 1:1 to obtain a compound of Comparative Example 2.

### Comparative Example 3

### (Synthesis of Compound A4)

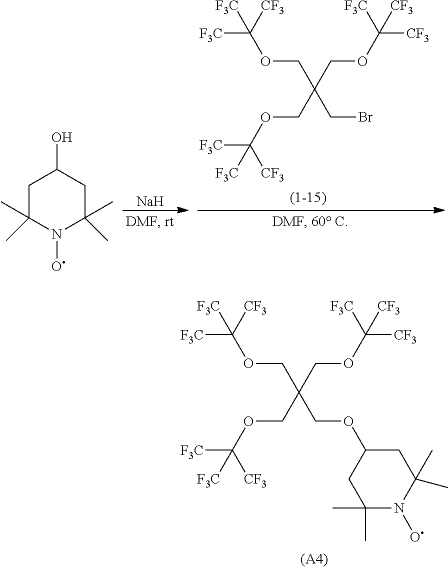

### Synthesis of 4-(3-((1,1,1,3,3,3-hexafluoro-2-(trifluoromethyl)propane-2-yl)oxy)-2,2-bis(((1,1,1,3,3,3-hexafluoro-2-(trifluoromethyl)propane-2-yl)oxy)methyl)propoxy)-2,2,6,6-tetramethylpiperidine-1-oxyl (A4)

In an argon stream, 10 mL of dimethylformamide (DMF) was added to 0.567 g (13.0 mmol) of 55% sodium hydride (NaH), and the mixture was stirred at room temperature for 10 minutes. 20 mL of a dimethylformamide solution containing 2.239 g (13.0 mmol) of 4-hydroxy-2,2,6,6-tetramethylpiperidine-1-oxyl was added dropwise thereto over 10 minutes, and the mixture was stirred at room temperature for 3 hours. Furthermore, 8.258 g (9.68 mmol) of 2-(3-bromo-2,2,-bis(((1,1,1,3,3,3-hexafluoro-2-(trifluoromethyl)propane-2-yl)oxy)methyl)propoxy)-1,1,1,3,3,3-hexafluoro-2-(trifluoromethyl)propane (1-15) synthesized through the above-described reaction and 60 mL of dimethylformamide were added thereto in an ice bath, and the mixture was stirred at 60° C. for 16 hours.

Water was added to the reaction solution, and extraction was performed with ethyl acetate. Thereafter, the extract was washed with a saturated sodium chloride aqueous solution, and the organic layer was dried with magnesium sulfate. The dried organic layer was concentrated under reduced pressure and then purified through silica gel column chromatography (hexane:ethyl acetate=9:1 to 4:1) to obtain target 4-(3-0,1,1,3,3,3-hexafluoro-2-(trifluoromethyl)propane-2-yl)oxy)-2,2-bis(((1,1,1,3,3,3-hexafluoro-2-(trifluoromethyl)propane-2-yl)oxy)methyl)propoxy)-2,2,6,6-tetramethylpiperidine-1-oxyl (A4) (yield of 7.503 g, percent yield of 83%).

When the obtained compound was subjected to mass spectrometry, a peak was confirmed at m/z=944 (M⁺). From this, it was confirmed that the synthesized compound is a compound represented by Formula (A4). In addition, the purity of the compound represented by Formula (A4) which was conformed through high-performance liquid chromatography (HPLC) was 98.1%.

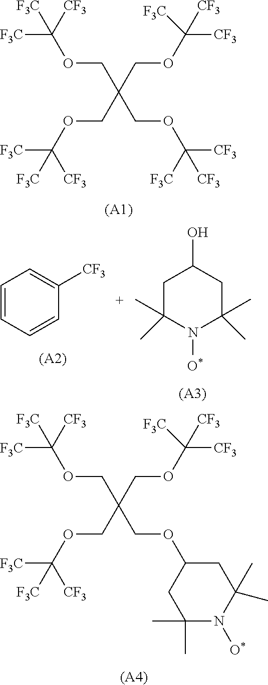

For the compounds of Examples 1 to 13 and Comparative Examples 1 to 3 thus obtained, the ¹⁹F spin-lattice relaxation time (T1) and the ¹⁹F spin-spin relaxation time (T2) were respectively measured through methods shown below. The results are shown in Table 1.

### (Measurement of ¹⁹F Spin-Lattice Relaxation Time (T1))

A compound was dissolved in a deuterated chloroform solution at a concentration of 5 mM, and the longitudinal relaxation time (T1) of ¹⁹F nucleus was measured through an inversion recovery method using a 500 MHz NMR device under the conditions shown below.

### (Measurement Conditions)

NMR device: JNM-ECA500 (manufactured by JEOL)
Measurement temperature: 36° C.
Pulse sequence: double_pulse
relaxation_delay: 10 [s]
tau_interval: 4, 3, 2, 1, 0.8, 0.6, 0.4, 0.2, 0.1[s], 80, 60, 40, 20, 10, 8, 6, 4, 2 [ms]
Number of times of integration: 128 times

### (Measurement of ¹⁹F Spin-Spin Relaxation Time (T2))

A compound was dissolved in a deuterated chloroform solution at a concentration of 5 mM, and the lateral relaxation time (T2) of ¹⁹F nucleus was measured through a Carr-Purcell-Meiboom-Gill (CPMG) method using a 500 MHz NMR device under the conditions shown below.

### (Measurement Conditions)

NMR device: JNM-ECA500 (manufactured by JEOL)
Measurement temperature: 36° C.
relaxation_delay: 10 [s]
tau_step: 1 [ms]
relaxation_delay: 1, 2, 3, 4, 5, 6, 8, 9, 10, 25, 50, 75 [ms], 0.10, 0.25, 0.50, 0.75, 1.00, 1.25, 1.50, 1.75, 2.00[s]
Number of times of integration: 128 times

TABLE 1

| | | T₁ | T₂ |
|---|---|---|---|
| | | 5 mM/CDCl₃ | 5 mM/CDCl₃ |
| | Compound | (sec) | (sec) |
| Example 1 | 11 | 0.033 | 0.025 |
| Example 2 | 12 | 0.035 | 0.026 |
| Example 3 | 13 | 0.068 | 0.050 |
| Example 4 | 14 | 0.188 | 0.140 |
| Example 5 | 15 | 0.052 | 0.043 |
| Example 6 | 16 | 0.052 | 0.042 |
| Example 7 | 17 | 0.055 | 0.046 |
| Example 8 | 18 | 0.121 | 0.095 |
| Example 9 | 19 | 0.303 | 0.228 |
| Example 10 | 20 | 0.025 | 0.019 |
| Example 11 | 21 | 0.024 | 0.019 |
| Example 12 | 22 | 0.048 | 0.036 |
| Example 13 | 23 | 0.144 | 0.108 |
| Comparative | A1 | 1.380 | 1.270 |
| Example 1 | | | |
| Comparative | A2, A3 | 1.041 | 0.880 |
| Example 2 | | | |
| Comparative | A4 | 0.077 | 0.057 |
| Example 3 | | | |

As shown in Table 1, the compounds of Examples 1 to 13 had shorter ¹⁹F spin-lattice relaxation time (T1) than the compounds of Comparative Examples 1 and 2.

In addition, the compound of Example 1, in which number of atoms in a chain structure through which nitroxide radical-containing groups represented by Formula (1) were bound to fluorine atom-containing groups represented by Formula (2) was 3, and the compound of Example 5 having three groups, each of which had the fluorine atom-containing group and was represented by Formula (4), had shorter ¹⁹F spin-lattice relaxation time (T1) than the compound of Comparative Example 3, in which the number of atoms in a chain structure through which a nitroxide radical-containing group represented by Formula (1) was bound to fluorine atom-containing groups represented by Formula (2) was 3 and which had three groups, each of which had the fluorine atom-containing group and was represented by Formula (4). It is presumed that this is because the compounds of Examples 1 and 5 and Comparative Example 3 have similar structures, but the compounds of Examples 1 and 5 had two nitroxide radical-containing groups represented by Formula (1) and the compound of Comparative Example 3 had only one nitroxide radical-containing group represented by Formula (1).

In addition, the compounds of Examples 1 to 13 had a ¹⁹F spin-spin relaxation time (T2) within an appropriate range. Compounds having a ¹⁹F spin-spin relaxation time (T2) of milliseconds or shorter have rapid signal decay and low sensitivity.

In addition, for the compounds of Example 1 (compound 11), Example 5 (compound 15), and Comparative Example 1 (compound A1), each 5 mM chloroform solution was adjusted, and a T1-weighted ¹⁹F-MRI image (phantom image) was obtained under the following imaging conditions.

### (Imaging Conditions)

Imaging device: MRI BioSpec117/11 (manufactured by Bruker)
Pulse sequence: RARE VTR
Repetition time: TR=300 ms
Echo time: TE=6 ms
Number of phase encodes=64
Number of echo trains=1
Flip angle=180°
Number of times of integration: 16 times
Total imaging time: 5.1 minutes

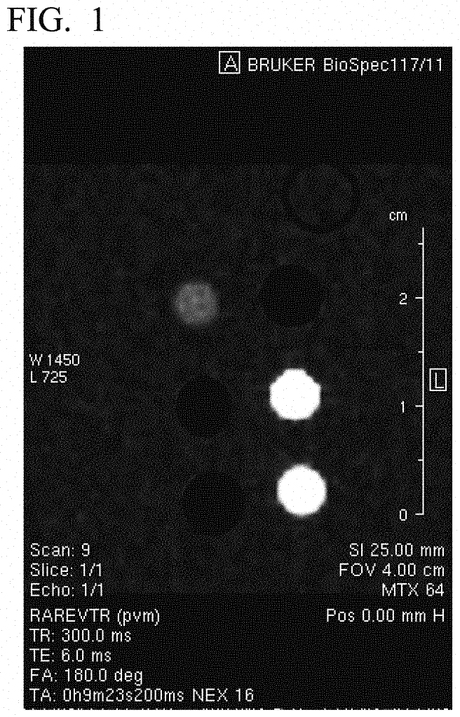

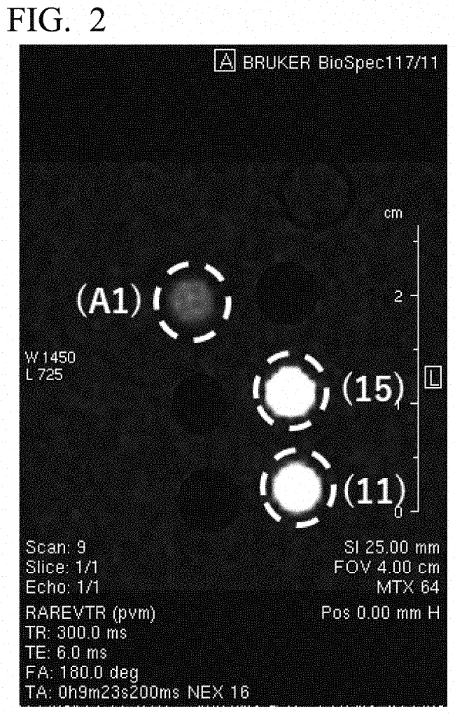

FIG. 1 is a ¹⁹F spin-lattice relaxation time (T1)-weighted ¹⁹F-MRI image for Example 1 (compound 11), Example 5 (compound 15), and Comparative Example 1 (compound A1). FIG. 2 is a ¹⁹F spin-lattice relaxation time (T1)-weighted ¹⁹F-MRI image for Example 1 (compound 11), Example 5 (compound 15), and Comparative Example 1 (compound A1) and a photograph in which positions of Examples 1 and 5 and Comparative Example 1 on the image shown in FIG. 1 are shown.

In addition, image processing software (ImageJ) was used to calculate a signal-to-noise ratio (SNR) of each of Examples 1 and 5 and Comparative Example 1 from gray values at positions of Examples 1 and 5 and Comparative Example 1 in the (T1)-weighted image shown in FIG. 1. The results are shown in Table 2.

TABLE 2

| | Compound | SNR |
|---|---|---|
| Example 1 | 11 | 29 |
| Example 5 | 15 | 42 |
| Comparative Example 1 | A1 | 6 |

As shown in FIGS. 1 and 2, the images of Example 1 (compound 11) and Example 5 (compound 15) were brighter than the image of Comparative Example 1 (compound A1).

In addition, as shown in Table 2, it was confirmed that larger SNRs are obtained in Example 1 (compound 11) and Example 5 (compound 15) than in Comparative Example 1 (compound A1) even at a short imaging time of about 5 minutes.

From these facts, it was shown that sufficiently clinically applicable images are obtained using Example 1 (compound 11) and Example 5 (compound 15) in contrast agents for MRI diagnosis using fluorine as a detection nucleus.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims

What is claimed is:

1. A fluorine-containing compound comprising:
two nitroxide radical-containing groups represented by General Formula (1) below; and
one to three fluorine atom-containing groups represented by Formula (2) below,
wherein the nitroxide radical-containing groups are bound to the fluorine atom-containing groups through a chain structure with 2 to 17 atoms linked by bonds between carbon atoms or through a chain structure with 2 to 17 atoms linked by an ether bond and bonds between carbon atoms, 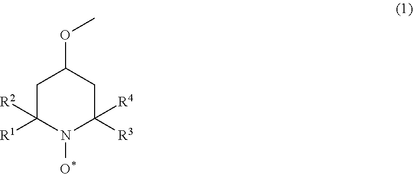
(in General Formula (1), R¹, R², R³, and R⁴ each independently represent a C1-10 alkyl group unsubstituted or substituted with a substituent containing no fluorine atoms),
—O—C(CF₃)₃ (2).

2. The fluorine-containing compound according to claim 1,
wherein two groups each having the nitroxide radical-containing group are bound to an identical carbon atom, and
the group having the nitroxide radical-containing group is represented by General Formula (3) below,
—CH₂—(O—(CH₂)ₛ)ₜ—R⁵ (3)
(in General Formula (3), s is an integer of 1 to 12, t is 0 or 1, and R⁵ is represented by General Formula (1) above).

3. The fluorine-containing compound according to claim 1,
wherein two or three groups each having the fluorine atom-containing group are bound to an identical carbon atom, and
the group having the fluorine atom-containing group is represented by Formula (4) below,
—CH₂—O—C(CF₃)₃ (4).

4. The fluorine-containing compound according to claim 1,
wherein the two groups each having the fluorine atom-containing group are bound to the carbon atom to which the two groups each having the nitroxide radical-containing group are bound.

5. The fluorine-containing compound according to claim 1,
wherein the carbon atom, to which the two groups each having the nitroxide radical-containing group are bound, is bound to the carbon atom, to which the three groups each having the fluorine atom-containing group are bound, through a linking group represented by Formula (5) below,
—CH₂—O— (5).

6. The fluorine-containing compound according to claim 1,
wherein R¹, R², R³, and R⁴ in General Formula (1) above each independently represent a methyl group or an ethyl group.

7. The fluorine-containing compound according to claim 1, which is used in a contrast agent for magnetic resonance imaging diagnosis using fluorine as a detection nucleus.

8. A contrast agent for magnetic resonance imaging diagnosis using fluorine as a detection nucleus, comprising:
the fluorine-containing compound according to claim 1.

9. The fluorine-containing compound according to claim 2,
wherein two or three groups each having the fluorine atom-containing group are bound to an identical carbon atom, and
the group having the fluorine atom-containing group is represented by Formula (4) below,
—CH₂—O—C(CF₃)₃ (4).

10. The fluorine-containing compound according to claim 2,
wherein the two groups each having the fluorine atom-containing group are bound to the carbon atom to which the two groups each having the nitroxide radical-containing group are bound.

11. The fluorine-containing compound according to claim 3,
wherein the two groups each having the fluorine atom-containing group are bound to the carbon atom to which the two groups each having the nitroxide radical-containing group are bound.

12. The fluorine-containing compound according to claim 2,
wherein the carbon atom, to which the two groups each having the nitroxide radical-containing group are bound, is bound to the carbon atom, to which the three groups each having the fluorine atom-containing group are bound, through a linking group represented by Formula (5) below,
—CH₂—O— (5).

13. The fluorine-containing compound according to claim 3,
wherein the carbon atom, to which the two groups each having the nitroxide radical-containing group are bound, is bound to the carbon atom, to which the three groups each having the fluorine atom-containing group are bound, through a linking group represented by Formula (5) below,
—CH₂—O— (5).

14. The fluorine-containing compound according to claim 2,
wherein R¹, R², R³, and R⁴ in General Formula (1) above each independently represent a methyl group or an ethyl group.

15. The fluorine-containing compound according to claim 3,
wherein R¹, R², R³, and R⁴ in General Formula (1) above each independently represent a methyl group or an ethyl group.

16. The fluorine-containing compound according to claim 4,
wherein R¹, R², R³, and R⁴ in General Formula (1) above each independently represent a methyl group or an ethyl group.

17. The fluorine-containing compound according to claim 5,
wherein R¹, R², R³, and R⁴ in General Formula (1) above each independently represent a methyl group or an ethyl group.

18. The fluorine-containing compound according to claim 2, which is used in a contrast agent for magnetic resonance imaging diagnosis using fluorine as a detection nucleus.

19. The fluorine-containing compound according to claim 3, which is used in a contrast agent for magnetic resonance imaging diagnosis using fluorine as a detection nucleus.

20. The fluorine-containing compound according to claim 4, which is used in a contrast agent for magnetic resonance imaging diagnosis using fluorine as a detection nucleus.
